(19) United States
(12) Patent Application Publication
(10) Pub. No.: US 2005/0239784 A1
(43) Pub. Date: Oct. 27, 2005

(54) 3-Phenyl analogs of toxoflavine as kinase inhibitors

(76) Inventors: Jean Fernand Armand LaCrampe, Le Mesnil-Esnard (FR); Richard William Connors, Harleysville, PA (US); Chih Yung Ho, Spring Howie, PA (US); Alan Ricardson, Buckingham (GB); Eddy Jean Edgard Freyne, Rumst (BE); Peter Jacobus Johannes Buijnsters, Breda (NL); Annette Cornelia Bakker, Borgerhout (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/520,768

(22) PCT Filed: Jul. 8, 2003
(86) PCT No.: PCT/EP03/50292

§ 371 Date: Jan. 10, 2005

(30) Foreign Application Priority Data
Jul. 15, 2002 (EP) 02077823.9

(51) Int. Cl. A61K 31/53; C07D 487/02

(52) U.S. Cl. 514/242; 544/184

(57) ABSTRACT

The present invention concerns the compounds of formula 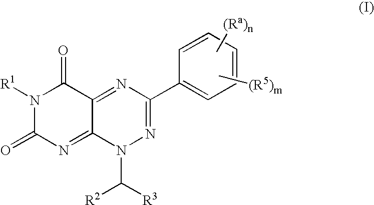
the N-oxide forms, the pharmaceutically acceptable addition salts and the stereo-chemically isomeric forms thereof, wherein n represents an integer being 0, 1 or 2; m represents an integer being 0 or 1; R represents C₁₋₄alkyl; R represents C₁₋₄alkyl; R³ represents C₁₋₄alkyl; or R² and R³ taken together with the carbon atom to which they are attached form a C₃₋₈cycloalkyl or Het¹ wherein said C₃₋₈cycloalkyl or Het¹ each independently may optionally be substituted with C₁₋₄alkyloxycarbonyl; R⁴ represents halo or C₁₋₄alkyloxy; R⁵ represents C₁₋₄alkyloxycarbonyl, —O-(mono- or di(C₁₋₄alkyl)aminosulfonyl), C₁₋₄alkyl substituted with one or where possible more substituent being selected from Het³ or NR⁶R⁷, C₁₋₄alkyloxy substituted with one or where possible more substituents being selected from amino, Het⁴ or NR⁸R⁹; R⁶ and R⁷ are each independently selected from hydrogen, C₁₋₄alkyl, C₁₋₄alkyloxyC₁₋₄alkyl, -Het⁵ or C₁₋₄alkyl substituted with one or where possible more substituents being selected from hydroxy, or Het⁵; R⁸ and R⁹ are each independently selected from hydrogen, C₁₋₄alkyl, -Het⁷ or mono- or di(C₁₋₄alkyl)aminosulphonyl; Het³ represents a heterocycle selected from piperidinyl, or piperazinyl wherein said monocyclic heterocycles each independently may optionally be substituted with one, or where possible two or three substituents each independently selected from hydroxy, aminosulfonyl, amino, mono- or di(C₁₋₄alkyl)aminosulfonyl, hydroxyC₁₋₄alkyloxyC₁₋₄alkyl or C₁₋₄alkyloxy; Het⁴ represents a heterocycle selected from morpholinyl, piperidinyl or piperazinyl wherein said monocyclic heterocycles each independently may optionally be substituted with one, or where possible two or three substituents each independently selected from C₁₋₄alkyl, C₁₋₄alkyloxycarbonyl or mono- or di(C₁₋₄alkyl)aminosulfonyl; Het⁵ represents a heterocycle selected from pyridinyl or piperidinyl wherein said monocyclic heterocycles each independently may optionally be substituted with one, or where possible two or three substituents each independently selected from aminosulfonyl, or mono- or di(C₁₋₄alkyl)aminosulfonyl; Het⁷ represents piperidinyl.

This invention relates to 1H-pyrimido[5.4-e][1,2,4]triazine-5,7-dione derivatives that inhibit cyclin-dependent serine/threonine kinases (Cdks), as well as kinases and phosphatases involved in cell cycle regulation such as the tyrosine kinases Wee1, Mik1 and Myt1 or the tyrosine dephosphatases such as Cdc25 and Pyp3. Cyclin-dependent kinases belong to the main regulators of cell division in eukaryotic organisms and their deregulation results in rearrangements, amplification and loss of chromosomes, events that are causally associated with cancer. As such these compounds are useful to treat cell proliferative disorders such as atherosclerosis, restenosis and cancer.

Cell cycle kinases are naturally occurring enzymes involved in regulation of the cell cycle (Meijer L., “Chemical Inhibitors of Cyclin-Dependent Kinases”, Progress in Cell Cycle Research, 1995; 1:35 1-363). Typical enzymes include serine/threonine kinases such as the cyclin-dependent kinases (cdk) cdk1, cdk2, cdk4, cdk5, cdk6 as well as tyrosine kinases such as AKT3 or Wee 1 kinase and tyrosine phosphatases such as cdc25 involved in cell cycle regulation. Increased activity or temporally abnormal activation or regulation of these kinases has been shown to result in development of human tumors and other proliferative disorders. Compounds that inhibit cdks, either by blocking the interaction between a cyclin and its kinase partner, or by binding to and inactivating the kinase, cause inhibition of cell proliferation, and are thus useful for treating tumors or other abnormally proliferating cells.

Several compounds that inhibit cdks have demonstrated preclinical anti-tumor activity. For example, flavopiridol is a flavonoid that has been shown to be a potent inhibitor of several types of breast and lung cancer cells (Kaur, et al., J. Natl. Cancer Inst., 1992; 84:1736-1740; Int. J. Oncol., 1996; 9:1143-1168). The compound has been shown to inhibit cdk2 and cdk4. Olomoucine [2-(hydroxyethylamino)-6-benzylamine-9-methylpurine] is a potent inhibitor of cdk2 and cdk5 (Vesely, et al., Eur. J. Biochem., 1994; 224:77 1-786); and has been shown to inhibit proliferation of approximately 60 different human tumor cell lines used by the National Cancer Institute (NCI) to screen for new cancer therapies (Abraham, et al., Biology of the Cell, 1995; 83: 105-120). More recently, flavonoid derivatives such toxoflavine (J. Chem. Soc. Perkin Trans. 1, 2001, 130-137) and 7-azapteridine derivatives (Japanese Unexamined Patent Application Laid Open H9-255681) have been disclosed as antineoplastic agents.

The toxoflavine derivatives of the present invention differ thereof in that the substituents at positions 1, 3 and 6 are modified with water solubility enhancing functionalities such as alcohol groups, aliphatic basic amine entities and aminosulphon(amine) substituents or a combination thereof, without loss of biological activity as anti-proliferative compounds.

Accordingly, the underlying problem to be solved by the present invention was to find further toxoflavine derivatives with an improved water solubility and concomitant cellular activity.

This invention concerns compounds of formula (I) 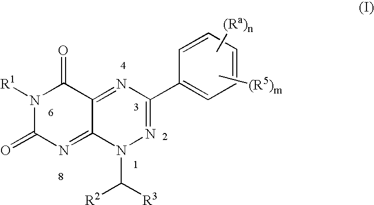
the N-oxide forms, the pharmaceutically acceptable addition salts and the stereo-chemically isomeric forms thereof, wherein
n represents an integer being 0, 1 or 2;
m represents an integer being 0 or 1
R¹ represents hydrogen, Ar¹, C₁₋₄alkyl or C₁₋₄alkyl substituted with morpholinyl or pyridinyl;
R² represents hydrogen, phenyl, C₁₋₄alkyl, C₁₋₄alkyloxycarbonyl or C₁₋₄alkyl substituted with hydroxy, phenyl or -oxy-halophenyl;
R³ represents hydrogen, phenyl, C₁₋₄alkyl, C₁₋₄alkyloxycarbonyl or C₁₋₄alkyl substituted with hydroxy, phenyl or -oxy-halophenyl; or
R² and R³ taken together with the carbon atom to which they are attached form a C₃₋₈cycloalkyl or Het¹ wherein said C₃₋₈cycloalkyl or Het¹ each independently may optionally be substituted with one, or where possible, two or three substituents each independently selected from C₁₋₄alkyloxycarbonyl, —C₁₋₄alkyl-Ar³ C₁₋₄alkylsulfonyl, aminosulfonyl, mono- or di(C₁₋₄alkyl)aminosulfonyl or —C(═NH)—NH₂;
R⁴ represents halo, nitro, hydroxy or C₁₋₄alkyloxy;
R⁵ represents formyl, hydroxy, cyano, phenyl, —O—Ar², NR⁶R⁷, C₁₋₄alkyl, C₁₋₄alkyloxy, C₁₋₄alkylsulfonyl, C₁₋₄alkylcarbonyl, C₁₋₄alkyloxycarbonyl, —O-(mono- or di(C₁₋₄alkyl)aminosulfonyl), Het², —SO₂-Het⁶, C₂₋₆alkenyl optionally substituted with phenyl,
   C₁₋₄alkyl substituted with one or where possible more substituent being selected from hydroxy, halo, Het³, NR⁶R⁷ or formyl,
   C₁₋₄alkyloxy substituted with one or where possible more substituents being selected from halo, amino, mono- or di(C₁₋₄alkyl)aminosulfonyl, aminosulfonyl, Het⁴, NR⁸R⁹ or —C(═O)-Het⁴;
R⁶ and R⁷ are each independently selected from hydrogen, C₁₋₄alkyl, C₁₋₄alkyloxyC₁₋₄alkyl, Het⁵ or C₁₋₄alkyl substituted with one or where possible more substituents being selected from hydroxy, Het⁵, C₁₋₄alkyloxycarbonyl, or C₁₋₄alkylsulfonyl;
R⁸ and R⁹ are each independently selected from hydrogen, C₁₋₄alkyl, C₁₋₄alkyloxycarbonyl, Het⁷, mono- or di(C₁₋₄alkyl)aminosulphonyl or aminosulphonyl;
Het¹ represents piperidinyl or dihydroindenyl;
Het² represents a heterocycle selected from piperidinyl, morpholinyl, or piperazinyl wherein said monocyclic heterocycles each independently may optionally be substituted with one, or where possible two or three substituents each independently selected from C₁₋₄alkyloxycarbonyl;
Het³ represents a heterocycle selected from morpholinyl, pyrrolidinyl, pyrrolyl, piperidinyl, or piperazinyl wherein said monocyclic heterocycles each independently may optionally be substituted with one, or where possible two or three substituents each independently selected from hydroxy, C₁₋₄alkyl, C₁₋₄alkyloxycarbonyl, hydroxyC₁₋₄alkyl, aminosulfonyl, NR¹⁰R¹¹, imidazolyl, tetrahydropyrimidinyl, amino, mono- or di(C₁₋₄alkyl)aminosulfonyl, hydroxyC₁₋₄alkyloxyC₁₋₄alkyl, C₁₋₄alkyloxyC₁₋₄alkyl or C₁₋₄alkyloxy;
R¹⁰ and R¹¹ are each independently selected from hydrogen, C₁₋₄alkyl, C₁₋₄alkyloxycarbonyl, aminosulfonyl, or mono- or di(C₁₋₄alkyl)aminosulfonyl;
Het⁴ represents a heterocycle selected from morpholinyl, piperidinyl, imidazolyl or piperazinyl wherein said monocyclic heterocycles each independently may optionally be substituted with one, or where possible two or three substituents each independently selected from hydroxy, C₁₋₄alkyl, C₁₋₄alkyloxycarbonyl, aminosulfonyl or mono- or di(C₁₋₄alkyl)-aminosulfonyl or Het⁴ represents a monovalent radical represented by formula (i); 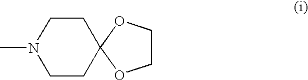
Het⁵ represents a heterocycle selected from pyridinyl, pyrimidinyl, pyrrolidinyl, or piperidinyl wherein said monocyclic heterocycles each independently may optionally be substituted with one, or where possible two or three substituents each independently selected from C₁₋₄alkyl, C₁₋₄alkyloxycarbonyl, aminosulfonyl, C₁₋₄alkylaminosulfonyl or mono- or di(C₁₋₄alkyl)aminosulfonyl;
Het⁶ represents morpholinyl;
Het⁷ represents pyridinyl, piperidinyl, piperazinyl or pyrimidinyl optionally substituted with C₁₋₄alkylphenyl, C₁₋₄alkyloxycarbonyl aminosulfonyl, or mono- or di(C₁₋₄alkyl)aminosulfonyl;
Ar¹ represents an aryl substituent selected from phenyl or naphthalenyl wherein said aryl substituents each independently may optionally be substituted with one, or where possibly two or three substituents each independently selected from nitro or C₁₋₄alkyloxycarbonyl;
Ar² represents phenyl optionally substituted with one or where possible two or three substituents each independently selected from the group consisting of halo and nitro;
Ar³ represents an aryl substituent selected from the group consisting of phenyl.

As used in the foregoing definitions and hereinafter, halo is generic to fluoro, chloro, bromo and iodo; C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like; C₁₋₆alkyl includes C₁₋₄alkyl and the higher homologues thereof having from 5 to 6 carbon atoms such as, for example, pentyl, hexyl, 3-methylbutyl, 2-methylpentyl and the like; C₁₋₁₂alkyl includes C₁₋₆alkyl and the higher homologues thereof having from 7 to 12 carbon atoms such as, for example, heptyl, octyl, nonyl, decyl and the like; C₁₋₄alkanediyl defines bivalent straight and branched chain saturated hydrocarbon radicals having from 1 to 4 carbon atoms such as, for example, methylene, 1,2-ethanediyl, 1,3-propanediyl, 1,4-butanediyl and the like; C₁₋₅alkanediyl includes C₁₋₄alkanediyl and the higher homologues thereof having 5 carbon atoms such as, for example, 1,5-pentanediyl and the like; C₁₋₆alkanediyl includes C₁₋₅alkanediyl and the higher homologues thereof having 6 carbon atoms such as, for example, 1,6-hexanediyl and the like; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like; C₂₋₆alkenediyl defines straight and branched chain hydrocarbon radicals containing one double bond and having from 2 to 6 carbon atoms such as, for example, ethenediyl, 2-propenediyl, 3-butenediyl, 2-pentenediyl, 3-pentenediyl, 3-methyl-2-butenediyl, and the like; haloC₁₋₄alkyl is defined as mono- or polyhalosubstituted C₁₋₄alkyl; C₁₋₆alkanediyl-oxy-C₁₋₆alkanediyl defines bivalent radicals of formula such as, for example, —CH₂—CH₂—O—CH₂—CH₂—, —CH₂—CH(CH₂CH₃)—O—CH(CH₃)—CH₂—, —CH(CH₃)—O—CH₂— and the like.

The pharmaceutically acceptable addition salts as mentioned hereinabove are meant to comprise the therapeutically active non-toxic acid addition salt forms which the compounds of formula (I) are able to form. The latter can conveniently be obtained by treating the base form with such appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The pharmaceutically acceptable addition salts as mentioned hereinabove are meant to comprise the therapeutically active non-toxic base addition salt forms which the compounds of formula (I) are able to form. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g. arginine, lysine.

Conversely said salt forms can be converted by treatment with an appropriate base or acid into the free acid or base form.

The term addition salt as used hereinabove also comprises the solvates which the compounds of formula (I) as well as the salts thereof, are able to form. Such solvates are for example hydrates, alcoholates and the like.

The term stereochemically isomeric forms as used hereinbefore defines the possible different isomeric as well as conformational forms which the compounds of formula (I) may possess. Unless otherwise mentioned or indicated, the chemical designation of compounds denotes the mixture of all possible stereochemically and conformationally isomeric forms, said mixtures containing all diastereomers, enantiomers and/or conformers of the basic molecular structure. All stereochemically isomeric forms of the compounds of formula (I) both in pure form or in admixture with each other are intended to be embraced within the scope of the present invention.

The N-oxide forms of the compounds of formula (I) are meant to comprise those compounds of formula (I) wherein one or several nitrogen atoms are oxidized to the so-called N-oxide, particularly those N-oxides wherein the piperidine-nitrogen is N-oxidized.

A preferred group of compounds consists of those compounds of formula (I) wherein one or more of the following restrictions apply:
R¹ represents C₁₋₄alkyl preferably methyl;
R² and R³ taken together with the carbon atom to which they are attached form a C₃₋₈cycloalkyl, preferably cyclopentyl or Het¹ wherein said C₃₋₈cycloalkyl or Het¹ each independently may optionally be substituted with one, or where possible, two or three substituents each independently selected from C₁₋₄alkyloxycarbonyl, —C₁₋₄alkyl-Ar³ or mono- or di(C₁₋₄alkyl)aminosulfonyl;
R⁴ represents halo preferably chloro or R⁴ represents C₁₋₄alkyloxy preferably methoxy;
R⁵ represents NR⁶R⁷, —O-(mono- or di(C₁₋₄alkyl)aminosulfonyl), -Het², —SO₂-Het⁶,
   C₁₋₄alkyl substituted with one or where possible more substituent being selected from Het³ or NR⁶R⁷,
   C₁₋₄alkyloxy substituted with one or where possible more substituents being selected from amino, Het⁴, or NR⁸R⁹;
R⁶ and R⁷ are each independently selected from hydrogen, C₁₋₄alkyl, C₁₋₄alkylsulfonyl, C₁₋₄alkyloxyC₁₋₄alkyl, Het⁵ or hydroxyC₁₋₄alkyl;
R⁸ and R⁹ are each independently selected from hydrogen, C₁₋₄alkyl, C₁₋₄alkyloxycarbonyl, Het⁷, or mono- or di(C₁₋₄alkyl)aminosulphonyl;
Het¹ represents piperidinyl or dihydroindenyl;
Het² represents morpholinyl;
Het³ represents a heterocycle selected from morpholinyl, pyrrolidinyl, pyrrolyl piperidinyl, or piperazinyl wherein said monocyclic heterocycles each independently may optionally be substituted with one, or where possible two or three substituents each independently selected from hydroxy, aminosulfonyl, mono- or di(C₁₋₄alkyl)aminosulfonyl or C₁₋₄alkyloxy;
Het⁴ represents a heterocycle selected from morpholinyl, piperidinyl, imidazolyl or piperazinyl wherein said monocyclic heterocycles each independently may optionally be substituted with one, or where possible two or three substituents each independently selected from hydroxy, C₁₋₄alkyl, C₁₋₄alkyloxycarbonyl, or mono- or di(C₁₋₄alkyl)aminosulfonyl, or Het⁴ represents a monovalent radical represented by formula (i); 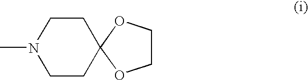
Het⁵ represents a heterocycle selected from pyridinyl or piperidinyl;
Het⁶ represents morpholinyl;
Het⁷ represents pyridinyl, or piperazinyl optionally substituted with C₁₋₄alkylphenyl, C₁₋₄alkyloxycarbonyl, or mono- or di(C₁₋₄alkyl)aminosulfonyl.

A group of interesting compounds consists of those compounds of formula (I) wherein one or more of the following restrictions apply:
R¹ represents Ar¹, C₁₋₄alkyl preferably methyl, or C₁₋₄alkyl substituted with morpholinyl;
R² represents hydrogen or C₁₋₄alkyl;
R³ represents hydrogen or C₁₋₄alkyl; or
R² and R³ taken together with the carbon atom to which they are attached form a C₃₋₈cycloalkyl or Het¹ wherein said C₃₋₈cycloalkyl or Het¹ each independently may optionally be substituted with C₁₋₄alkyloxycarbonyl;
R⁴ represents halo preferably chloro or R⁴ represents C₁₋₄alkyloxy preferably methoxy;
R⁵ represents C₁₋₄alkyloxycarbonyl, oxy-(mono- or di(C₁₋₄alkyl)aminosulfonyl),
   C₁₋₄alkyl substituted with one or where possible more substituent being selected from Het³ or NR⁶R⁷,
   C₁₋₄alkyloxy substituted with one or where possible more substituents being selected from amino, Het⁴ or NR⁸R⁹;
R⁶ and R⁷ are each independently selected from hydrogen, C₁₋₄alkyl,
   C₁₋₄alkyloxyC₁₋₄alkyl, Het⁵ or C₁₋₄alkyl substituted with one or where possible more substituents being selected from hydroxy or Het⁵;
R⁸ and R⁹ are each independently selected from hydrogen, C₁₋₄alkyl,
   C₁₋₄alkyloxycarbonyl, Het⁷ or mono- or di(C₁₋₄alkyl)aminosulphonyl;
Het¹ represents piperidinyl;
Het³ represents a heterocycle selected from morpholinyl, pyrrolidinyl, piperidinyl, or piperazinyl wherein said monocyclic heterocycles each independently may optionally be substituted with one, or where possible two or three substituents each independently selected from hydroxy, C₁₋₄alkyl, aminosulfonyl, amino, mono- or di(C₁₋₄alkyl)aminosulfonyl, hydroxyC₁₋₄alkyloxyC₁₋₄alkyl or C₁₋₄alkyloxy;
Het⁵ represents pyridinyl optionally substituted with mono- or di(C₁₋₄alkyl)aminosulfonyl;
Het⁷ represents piperidinyl optionally substituted with C₁₋₄alkylphenyl, C₁₋₄alkyloxycarbonyl, or mono- or di(C₁₋₄alkyl)aminosulfonyl;
Ar¹ represents an aryl substituent selected from phenyl or naphthalenyl.

A further group of interesting compounds consists of those compounds of formula (I) wherein one or more of the following restrictions apply:
R¹ represents C₁₋₄alkyl preferably methyl;
R² and R³ each independently represent C₁₋₄alkyl preferably methyl;
R² and R³ taken together with the carbon atom to which they are attached form a C₃₋₈cycloalkyl, preferably cyclopentyl or Het¹ preferably piperidinyl optionally substituted with C₁₋₄alkyloxycarbonyl preferably t-butyloxycarbonyl;
R⁴ represents C₁₋₄alkyloxy preferably methoxy;
R⁵ represents C₁₋₄alkyloxy, C₁₋₄alkyloxycarbonyl, oxy-(mono- or di(C₁₋₄alkyl)aminosulfonyl), C₁₋₄alkyl substituted with one or where possible more substituent being selected from Het³ or NR⁶R⁷, or Het⁵ represents C₁₋₄alkyloxy substituted with one or where possible more substituents being selected from amino, mono- or di(C₁₋₄alkyl)aminosulfonyl, aminosulfonyl or Het⁴;
R⁶ and R⁷ are each independently selected from hydrogen, C₁₋₄alkyl,
   C₁₋₄alkyloxyC₁₋₄alkyl, Het⁵ or C₁₋₄alkyl substituted with one or where possible more substituents being selected from hydroxy, or Het⁵;
R⁸ and R⁹ are each independently selected from hydrogen, C₁₋₄alkyl, C₁₋₄alkyloxycarbonyl, Het⁷, mono- or di(C₁₋₄alkyl)aminosulphonyl or aminosulphonyl;
Het³ represents a heterocycle selected from pyrrolidinyl, piperidinyl, or piperazinyl wherein said monocyclic heterocycles each independently may optionally be substituted with one, or where possible two or three substituents each independently selected from hydroxy, C₁₋₄alkyloxycarbonyl, aminosulfonyl, amino, mono- or di(C₁₋₄alkyl)aminosulfonyl, hydroxyC₁₋₄alkyloxyC₁₋₄alkyl, or C₁₋₄alkyloxy;
Het⁴ represents a heterocycle selected from morpholinyl, piperidinyl, imidazolyl or piperazinyl wherein said monocyclic heterocycles each independently may optionally be substituted with one, or where possible two or three substituents each independently selected from C₁₋₄alkyl, C₁₋₄alkyloxycarbonyl, or mono- or di(C₁₋₄alkyl)aminosulfonyl;
Het⁵ represents a heterocycle selected from pyrimidinyl or piperidinyl wherein said monocyclic heterocycles each independently may optionally be substituted with one, or where possible two or three substituents each independently selected from C₁₋₄alkyl, or mono- or di(C₁₋₄alkyl)aminosulfonyl;
Het⁷ represents pyridinyl, piperidinyl, piperazinyl or pyrimidinyl optionally substituted with C₁₋₄alkylphenyl, C₁₋₄alkyloxycarbonyl aminosulfonyl, or mono- or di(C₁₋₄alkyl)aminosulfonyl.

Also of interest, are the group of compounds of formula (I) wherein one or more of the following restrictions apply:
R¹ represents C₁₋₄alkyl preferably methyl
R² represents hydrogen, C₁₋₄alkyl or C₁₋₄alkyl substituted with phenyl;
R³ represents hydrogen, C₁₋₄alkyl or C₁₋₄alkyl substituted with phenyl; or
R² and R³ taken together with the carbon atom to which they are attached form a C₃₋₈cycloalkyl or Het¹ wherein said C₃₋₈cycloalkyl or Het¹ each independently may optionally be substituted with one, or where possible, two or three substituents each independently selected from C₁₋₄alkyloxycarbonyl or —C₁₋₄alkyl-Ar³;
R⁴ represents halo or C₁₋₄alkyloxy preferably methoxy;
R⁵ represents NR⁶R⁷, C₁₋₄alkyloxycarbonyl, —O-(mono- or di(C₁₋₄alkyl)aminosulfonyl),
   C₁₋₄alkyl substituted with one or where possible more substituent being selected from Het³ or NR⁶R⁷,
   C₁₋₄alkyloxy substituted with one or where possible more substituents being selected from Het⁴ or NR⁸R⁹;
R⁶ and R⁷ are each independently selected from hydrogen, C₁₋₄alkyl,
   C₁₋₄alkyloxyC₁₋₄alkyl, Het⁵ or C₁₋₄alkyl substituted with one or where possible more substituents being selected from hydroxy or Het⁵;
R⁸ and R⁹ are each independently selected from hydrogen or C₁₋₄alkyl;
Het¹ represents piperidinyl;
Het³ represents a heterocycle selected from morpholinyl, piperidinyl, or piperazinyl;
Het⁴ represents a heterocycle selected from morpholinyl or piperazinyl wherein said monocyclic heterocycles each independently may optionally be substituted with C₁₋₄alkyloxycarbonyl;
Het⁵ represents a heterocycle selected from pyridinyl or piperidinyl wherein said monocyclic heterocycles each independently may optionally be substituted with one, or where possible two or three substituents each independently selected from aminosulfonyl or mono- or di(C₁₋₄alkyl)aminosulfonyl;
Ar³ represents phenyl.

A remarkable group of compounds are those according to formula (I) wherein one or more of the following restrictions apply;
n represents 2;
R¹ represents hydrogen, Ar¹, C₁₋₄alkyl or C₁₋₄alkyl substituted with morpholinyl or pyridinyl;
R² represents hydrogen, phenyl or C₁₋₄alkyl optionally substituted with hydroxy or phenyl;
R³ represents hydrogen, phenyl or C₁₋₄alkyl optionally substituted with hydroxy or phenyl; or
R⁴ represents halo preferably halo, or R⁴ represents C₁₋₄alkyloxy preferably methoxy;
R⁵ represents cyano, phenyl, —O—Ar², C₁₋₄alkyl, C₁₋₄alkyloxy, C₁₋₄alkyloxycarbonyl,
   C₂₋₆alkenyl optionally substituted with phenyl,
   C₁₋₄alkyl substituted with halo preferably trifluoromethyl,
   C₁₋₄alkyloxy substituted with halo preferably chloro or fluoro;
R⁶ and R⁷ are each independently selected from hydrogen, C₁₋₄alkyl,
   C₁₋₄alkyloxyC₁₋₄alkyl, Het⁵ or C₁₋₄alkyl substituted with one or where possible more substituents being selected from hydroxy, Het⁵, C₁₋₄alkyloxycarbonyl, or C₁₋₄alkylsulfonyl.

It is also an embodiment of the present invention to provide a group of compounds of formula (I) wherein one or more of the following restrictions apply;
R¹ represents C₁₋₄alkyl preferably methyl;
R² represents hydrogen, phenyl, C₁₋₄alkyl, C₁₋₄alkyloxycarbonyl or C₁₋₄alkyl substituted with phenyl;
R³ represents hydrogen, phenyl, C₁₋₄alkyl, C₁₋₄alkyloxycarbonyl or C₁₋₄alkyl substituted with phenyl; or
R² and R³ taken together with the carbon atom to which they are attached form a C₃₋₈cycloalkyl or Het¹ wherein said C₃₋₈cycloalkyl or Het¹ each independently may optionally be substituted with one, or where possible, two or three substituents each independently selected from C₁₋₄alkyloxycarbonyl, or —C₁₋₄alkyl-Ar³;
R⁴ represents halo or C₁₋₄alkyloxy;
R⁵ represents NR⁶R⁷, —O-(mono- or di(C₁₋₄alkyl)aminosulfonyl), -Het²,
   C₁₋₄alkyl substituted with one or where possible more substituent being selected from Het³ or NR⁶R⁷,
   C₁₋₄alkyloxy substituted with one or where possible more substituents being selected from amino, Het⁴, or NR⁸R⁹;
R⁶ and R⁷ are each independently selected from hydrogen, C₁₋₄alkyl, C₁₋₄alkyloxyC₁₋₄alkyl, Het⁵ or C₁₋₄alkyl substituted with one or where possible more substituents being selected from hydroxy or C₁₋₄alkylsulfonyl;
R⁸ and R⁹ are each independently selected from hydrogen, C₁₋₄alkyl,
   C₁₋₄alkyloxycarbonyl, Het⁷ or mono- or di(C₁₋₄alkyl)aminosulphonyl;
Het² represents morpholinyl;
Het³ represents a heterocycle selected from morpholinyl, pyrrolidinyl, piperidinyl, or piperazinyl wherein said monocyclic heterocycles each independently may optionally be substituted with one, or where possible two or three substituents each independently selected from hydroxy, C₁₋₄alkyl, aminosulfonyl, mono- or di(C₁₋₄alkyl)aminosulfonyl or C₄alkyloxy;
Het⁴ represents a heterocycle selected from morpholinyl, piperidinyl, imidazolyl or piperazinyl wherein said monocyclic heterocycles each independently may optionally be substituted with one, or where possible two or three substituents each independently selected from hydroxy, C₁₋₄alkyl, C₁₋₄alkyloxycarbonyl, aminosulfonyl or mono- or di(C₁₋₄alkyl)aminosulfonyl or Het⁴ represents a monovalent radical represented by formula (i); 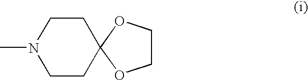
Het⁵ represents a heterocycle selected from pyridinyl or piperidinyl wherein said monocyclic heterocycles each independently may optionally be substituted with mono- or di(C₁₋₄alkyl)aminosulfonyl;
Het⁷ represents piperidinyl optionally substituted with C₁₋₄alkylphenyl;
Ar³ represents phenyl,

A remarkable group of compounds are those according to formula (I) wherein one or more of the following restrictions apply;
R¹ represents C₁₋₄alkyl preferably methyl;
R² represents C₁₋₄alkyl preferably methyl;
R³ represents C₁₋₄alkyl preferably methyl; or
R² and R³ taken together with the carbon atom to which they are attached form a C₃₋₈cycloalkyl preferably cyclopentyl or Het¹ preferably piperidinyl wherein said C₃₋₈cycloalkyl or Het¹ each independently may optionally be substituted with C₁₋₄alkyloxycarbonyl preferably t-butoxycarbonyl;
R⁴ represents halo or C₁₋₄alkyloxy;
R⁵ represents C₁₋₄alkyloxycarbonyl, —O-(mono- or di(C₁₋₄alkyl)aminosulfonyl),
   C₁₋₄alkyl substituted with one or where possible more substituent being selected from Het³ or NR⁶R⁷,
   C₁₋₄alkyloxy substituted with one or where possible more substituents being selected from amino, Het⁴ or NR⁸R⁹;
R⁶ and R⁷ are each independently selected from hydrogen, C₁₋₄alkyl,
   C₁₋₄alkyloxyC₁₋₄alkyl, -Het⁵ or C₁₋₄alkyl substituted with one or where possible more substituents being selected from hydroxy, or Het⁵;
R⁸ and R⁹ are each independently selected from hydrogen, C₁₋₄alkyl, -Het⁷ or mono- or di(C₁₋₄alkyl)aminosulphonyl;
Het³ represents a heterocycle selected from piperidinyl, or piperazinyl wherein said monocyclic heterocycles each independently may optionally be substituted with one, or where possible two or three substituents each independently selected from hydroxy, aminosulfonyl, amino, mono- or di(C₁₋₄alkyl)aminosulfonyl, hydroxyC₁₋₄alkyloxyC₁₋₄alkyl or
   C₁₋₄alkyloxy;
Het⁴ represents a heterocycle selected from morpholinyl, piperidinyl or piperazinyl wherein said monocyclic heterocycles each independently may optionally be substituted with one, or where possible two or three substituents each independently selected from C₁₋₄alkyl,
   C₁₋₄alkyloxycarbonyl or mono- or di(C₁₋₄alkyl)aminosulfonyl;
Het⁵ represents a heterocycle selected from pyridinyl or piperidinyl wherein said monocyclic heterocycles each independently may optionally be substituted with one, or where possible two or three substituents each independently selected from aminosulfonyl, or mono- or di(C₁₋₄alkyl)aminosulfonyl;
Het⁷ represents piperidinyl.

A further group of compounds are those according to formula (I) wherein one or more of the following restrictions apply;
R¹ represents C₁₋₄alkyl preferably methyl;
R² represents hydrogen, C₁₋₄alkyl preferably methyl or isopropyl, or R² represents C₁₋₄alkyl substituted with hydroxy, preferably hydroxy-ethyl-;
R³ represents hydrogen, phenyl, C₁₋₄alkyl preferably methyl, C₁₋₄alkyloxycarbonyl preferably methoxycarbonyl or C₁₋₄alkyl substituted with phenyl;
R² and R³ taken together with the carbon atom to which they are attached form a C₃₋₈cycloalkyl preferably C₅₋₈cycloalkyl or Het¹ wherein said C₃₋₈cycloalkyl or Het¹ each independently may optionally be substituted with C₁₋₄alkyloxycarbonyl preferably t-butoxycarbonyl, —C₁₋₄alkyl-Ar³ or mono- or di(C₁₋₄alkyl)aminosulfonyl preferably dimethylaminosulfonyl;
R⁴ represents halo preferably chloro or C₁₋₄alkyloxy;
R⁵ represents hydroxy, —O—Ar², C₁₋₄alkyloxycarbonyl, Het², C₁₋₄alkyl substituted with Het³ or NR⁶R⁷, or R⁵ represents C₁₋₄alkyloxy substituted with Het⁴;
R⁶ and R⁷ are each independently selected from the hydrogen, C₁₋₄alkyl or C₁₋₄alkyl substituted with hydroxy;
Het³ represents a heterocycle selected from morpholinyl, pyrrolidinyl, piperidinyl or piperazinyl optionally substituted with one or two substituents each independently selected from hydroxy, C₁₋₄alkyl, or C₁₋₄alkyloxycarbonyl preferably t-butyl-oxycarbonyl-;
Het⁴ represents a heterocycle selected from morpholinyl, piperidinyl or piperazinyl wherein said heterocycles each independently may optionally be substituted with one, or where possible two or three C₁₋₄alkyl substituent or Het⁴ represents a monovalent radical represented by formula (i); or
Ar² represents phenyl optionally substituted with one or where possible two or three halo substituents, preferably chloro;

Other special group of compounds are;
those compounds of formula (I) wherein m represents 1 and R⁵ is in the para position relative to the carbon atom bearing the phenyl substituent;
those compounds of formula (I) wherein R¹ is methyl;
those compounds of formula (I) wherein R² and R³ taken together with the carbon atom to which they are attached form a C₃₋₈cycloalkyl, preferably cyclopentyl;
those compounds of formula (I) wherein R² and R³ taken together with the carbon atom to which they are attached form piperidinyl optionally substituted with C₁₋₄alkyloxycarbonyl preferably t-butoxycarbonyl;
those compounds of formula (I) wherein R² and R³ each represents a C₁₋₄alkyl, preferably methyl;
those compounds of formula (I) wherein R² and R³ each independently represents phenyl or —CH₂-phenyl;
those compounds of formula (I) wherein Het³ represent a heterocycle selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent.
those compounds of formula (I) wherein R⁵ represents formyl, hydroxy, cyano, phenyl,
—O—Ar², NR⁶R⁷, C₁₋₄alkylsulfonyl, C₁₋₄alkylcarbonyl, C₁₋₄alkyloxycarbonyl, —O-(mono- or di(C₁₋₄alkyl)aminosulfonyl), Het², —SO₂-Het⁶, C₂₋₆alkenyl optionally substituted with phenyl,
C₁₋₄alkyl substituted with one or where possible more substituent being selected from hydroxy, halo, Het³, NR⁶R⁷ or formyl, or C₁₋₄alkyloxy substituted with one or where possible more substituents being selected from halo, amino, mono- or di(C₁₋₄alkyl)-aminosulfonyl, aminosulfonyl, Het⁴, NR⁸R⁹ or —C(—O)-Het⁴;
those compounds of formula (I) with R⁵ being a C₁₋₄alkyloxy said C₁₋₄alkyloxy being substituted with one Het⁴ substituent with Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.
those compounds of formula (I) with R⁵ being a C₁₋₄alkyloxy said C₁₋₄alkyloxy being substituted with one Het⁴ substituent with Het⁴ being selected from the group consisting of piperidinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.
those compounds of formula (I) with R⁵ being NR⁶R⁷ wherein either R⁶ or R⁷ represents
C₁₋₄alkylsulfonyl or C₁₋₄alkylcarbonyl, preferably methylsulfonyl or methylcarbonyl.
those compounds of formula (I) with R⁵ being C₂₋₆alkenyl said alkenyl being substituted with phenyl.
those compounds of formula (I) wherein R⁵ represents hydrogen and R⁴ represents halo, preferably chloro.

In order to simplify the structural representation of the compounds of formula (I), the group 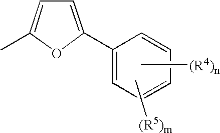
will hereinafter be represented by the symbol Q.

The compounds of this invention can be prepared by any of several standard synthetic processes commonly used by those skilled in the art of organic chemistry and described for instance in the following references; “Heterocyclic Compounds”—Vol. 24 (part4) p 261-304 Fused pyrimidines, Wiley—Interscience; Chem. Pharm. Bull., Vol 41(2) 362-368 (1993); J. Chem. Soc., Perkin Trans. 1, 2001, 130-137.

As further exemplified in the experimental part of the description, the compounds of formula (I) were generally prepared using three alternative synthesis schemes. In a first alternative, the compounds of formula (I) were prepared by nitrosative cyclisation of intermediates of formula (II) with NaNO₂ in acetic acid (AcOH). The thus obtained azapteridines comprising the 5-nitroso intermediates of formula (III) are subsequently converted in the final compounds with formula (I) by refluxing the mixture in for example acetic anhydride or ethanol (EtOH) comprising dithiothreitol (DTT).

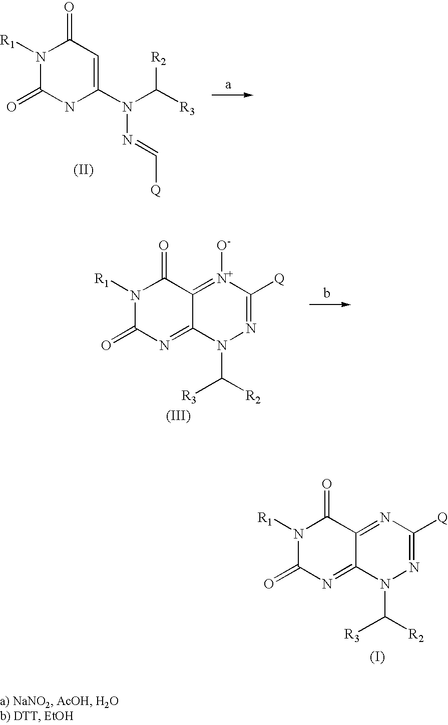

Alternatively, the intermediates of formula (III) are dealkylated by heating in N,N-Dimethylformamide (DMF) at temperatures ranging from 90-150° C. for 3-6 hours. The thus obtained reumycin derivatives of formula (IV) are subsequently alkylated in 1,4-dioxane further comprising an appropriate base such as anhydrous potassium carbonate, sodium hydride or sodium hydrogen carbonate, preferably anhydrous potassium carbonate and an alkylating agent such as dialkylsulfate, alkyliodide or alkylbromide, preferably alkylbromide, yielding the final compounds of formula (I).

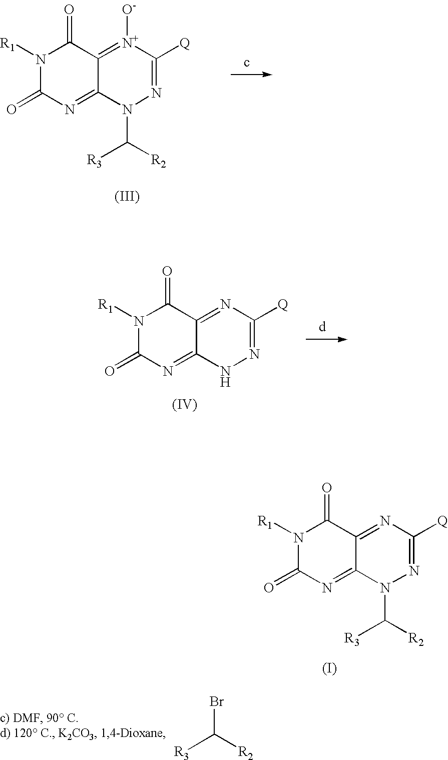

In the aforementioned reaction schemes, the substituted imines or Schiffs bases of formula (II) can generally be prepared by reacting a primary amine of formula (V) with an aldehyde of formula (VI) in a traditional condensation reaction using amongst others ethanol as a suitable solvent.

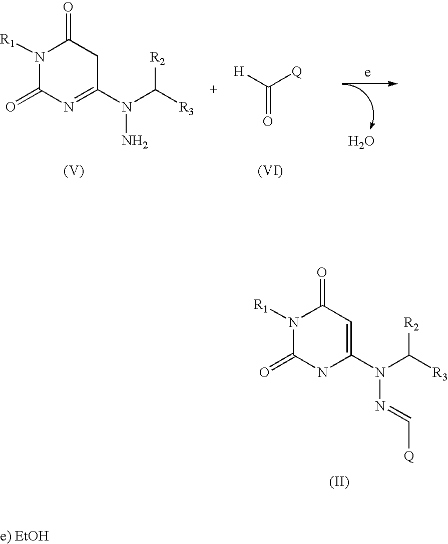

Finally, as an alternative to the above, the compounds of formula (I) can be prepared in a condensation reaction between a primary amine of formula (Va) with an aldehyde of formula (VI) using amongst others, ethanol as a suitable solvent.

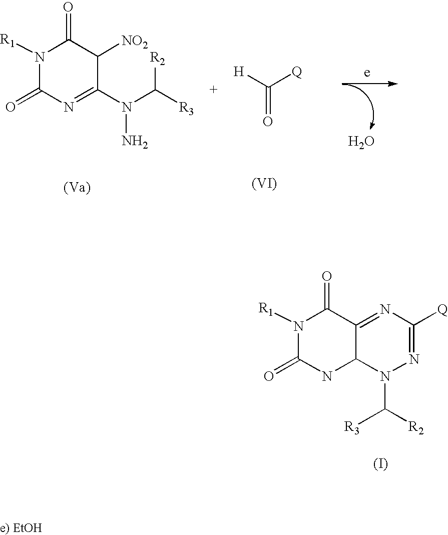

The intermediates of formula (V) and (Va) were generally prepared as depicted in reaction scheme 1.

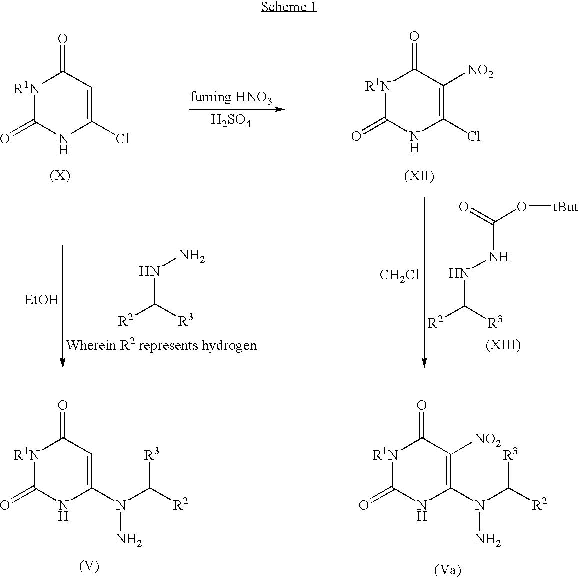

In order to introduce further R2 substituents the urea derivative of formula (XI) was shielded with the protective group t-butoxycarbonyl. This is introduced by treating a ketone of formula formula (XIV) with t-butoxycarbonylhydrazine and subsequent reduction with Pt/C/H₂ in EtOH or by the slow addition of NaBH₄ in THF.

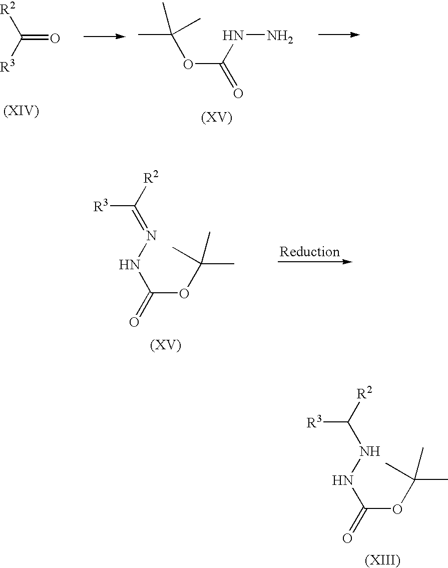

The protecting group is easily removed by treating the protected amine with trifluoroacetic acid (TFA) in CH₂Cl₂ as a solvent.

As depicted in scheme 2, art known techniques such as described in “Introduction to is Organic Chemistry”—A. Streitweiser, second ed. Macmillan Publishing Inc. p 1104, were used to prepare the pyrimidines of formula (IX). In general, the synthesis of said pyrimidines consists of a condensation between 1,3-dicarbonyl compounds such as diethylpropanedioate and a material containing the general structure N—C—N such as urea and the compounds of formula (VIII). The urea compounds of formula (VIII) are prepared using art know techniques, in particular the reaction of isocyanates such as benzoylisocyanate with an amine such as represented by formula (VII). In this particular reaction scheme, the benzoyl substituent is released from the urea complex of formula (VIIIa) by hydratation with water.

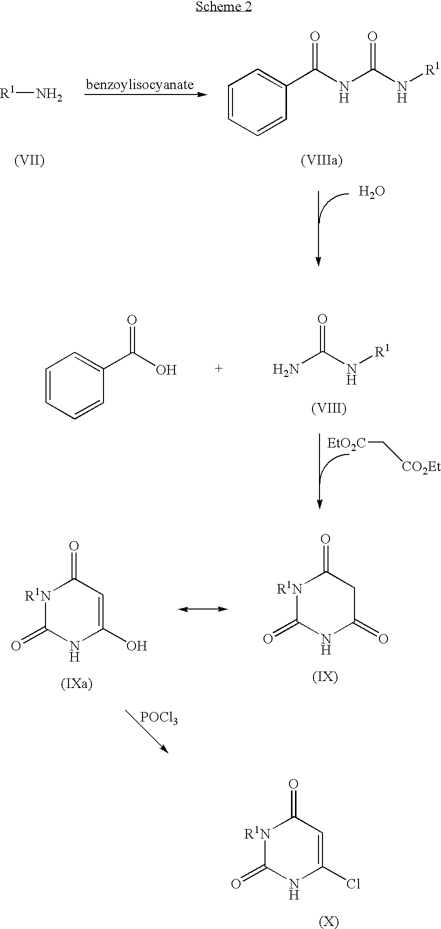

In a final step the tautomeric form of the thus obtained pyrimidines (IXa) were halogenated using an appropriate halogenating agent such as SOCl₂, POCl₃, PCl₅ or PBr₃.

Some of the starting aldehydes of formula (VI) were described in the literature. The others were prepared according to known procedures. For instance, starting from the commercially available 4-Hydroxybenzaldehyde (VI-a), we prepared the different aldehydes (VI-b) by a Mitsunobu reaction using the corresponding amino-alcohol. Then, according to the previously described scheme, we synthesized the respective compounds of formula (I);

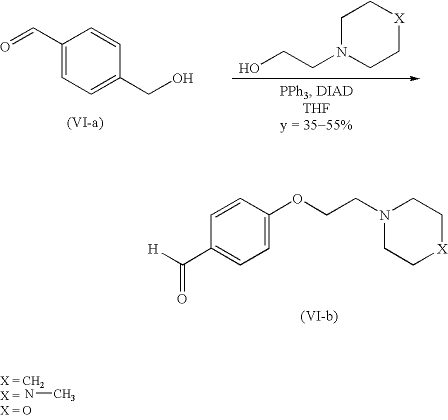

Where necessary or desired, any one or more of the following further steps in any order may be performed:
(i) removing any remaining protecting group(s);
(ii) converting a compound of formula (I) or a protected form thereof into a further compound of formula (I) or a protected form thereof;
(iii) converting a compound of formula (I) or a protected form thereof into a N-oxide, a salt, a quaternary amine or a solvate of a compound of formula (I) or a protected form thereof;
(iv) converting a N-oxide, a salt, a quaternary amine or a solvate of a compound of formula (I) or a protected form thereof into a compound of formula (I) or a protected form thereof;
   (v) converting a N-oxide, a salt, a quaternary amine or a solvate of a compound of formula (I) or a protected form thereof into another N-oxide, a pharmaceutically acceptable addition salt a quaternary amine or a solvate of a compound of formula (I) or a protected form thereof;
   (vi) where the compound of formula (I) is obtained as a mixture of (R) and (S) enantiomers resolving the mixture to obtain the desired enantiomer.

Compounds of formula (I), N-oxides, addition salts, quaternary amines and stereochemical isomeric forms thereof can be converted into further compounds according to the invention using procedures known in the art, for example:

It will be appreciated by those skilled in the art that in the processes described above the functional groups of intermediate compounds may need to be blocked by protecting groups.

Functional groups which it is desirable to protect include hydroxy, amino and carboxylic acid. Suitable protecting groups for hydroxy include trialkylsilyl groups (e.g. tert-butyldimethylsilyl, tert-butyldiphenylsilyl or trimethylsilyl), benzyl and tetrahydro-pyranyl. Suitable protecting groups for amino include tert-butyloxycarbonyl or benzyloxycarbonyl. Suitable protecting groups for carboxylic acid include C₍₁₋₆₎alkyl or benzyl esters.

The protection and deprotection of functional groups may take place before or after a reaction step.

The use of protecting groups is fully described in ‘Protective Groups in Organic Chemistry’, edited by J W F McOmie, Plenum Press (1973), and ‘Protective Groups in Organic Synthesis’ 2^{nd} edition, T W Greene & P G M Wutz, Wiley Interscience (1991).

Additionally, the N-atoms in compounds of formula (I) can be methylated by art-known methods using CH₃—I in a suitable solvent such as, for example 2-propanone, tetrahydrofuran or dimethylformamide.

The compounds of formula (I) can also be converted into each other following art-known procedures of functional group transformation of which some examples are mentioned hereinabove.

The compounds of formula (I) may also be converted to the corresponding N-oxide forms following art-known procedures for converting a trivalent nitrogen into its N-oxide form. Said N-oxidation reaction may generally be carried out by reacting the starting material of formula (I) with 3-phenyl-2-(phenylsulfonyl)oxaziridine or with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. t-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

Pure stereochemically isomeric forms of the compounds of formula (I) may be obtained by the application of art-known procedures. Diastereomers may be separated by physical methods such as selective crystallization and chromatographic techniques, e.g. counter-current distribution, liquid chromatography and the like.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography, in particular liquid chromatography using a chiral stationary phase.

Some of the intermediates and starting materials as used in the reaction procedures mentioned hereinabove are known compounds and may be commercially available or may be prepared according to art-known procedures.

The compounds of the present invention are useful because they possess pharmacological properties. They can therefore be used as medicines.

As described in the experimental part hereinafter, the growth inhibitory effect and anti-tumor activity of the present compounds has been demonstrated in vitro, in enzymatic assays on kinases and phosphatases involved in cell cycle regulation. Anti-tumor activity was also demonstrated in vitro, in a cell based assay comprising contacting the cells with the compounds and assessing the effect of AKT3 on MAPK phosphorylation. In an alternative assay, the growth inhibitory effect of the compounds was tested on the ovarian carcinoma cell line A2780 using art known cytotoxicity assays such as LIVE/DEAD (Molecular Probes) MTT.

Accordingly, the present invention provides the compounds of formula (I) and their pharmaceutically acceptable N-oxides, addition salts, quaternary amines and stereochemically isomeric forms for use in therapy. More particular in the treatment or prevention of T cell mediated diseases. The compounds of formula (I) and their pharmaceutically acceptable N-oxides, addition salts, quaternary amines and the stereochemically isomeric forms may hereinafter be referred to as compounds according to the invention.

Disorders for which the compounds according to the invention are particularly useful are atherosclerosis, restinosis and cancer.

In view of the utility of the compounds according to the invention, there is provided a method for the treatment of an animal, for example, a mammal including humans, suffering from a cell proliferative disorder such as atherosclerosis, restinosis and cancer, which comprises administering an effective amount of a compound according to the present invention.

In yet a further aspect, the present invention provides the use of the compounds according to the invention in the manufacture of a medicament for treating any of the aforementioned cell proliferative disorders or indications.

The amount of a compound according to the present invention, also referred to here as the active ingredient, which is required to achieve a therapeutical effect will be, of course, vary with the particular compound, the route of administration, the age and condition of the recipient, and the particular disorder or disease being treated. A suitable daily dose would be from 0.01 mg/kg to 50 mg/kg body weight, in particular from 0.05 mg/kg to 10 mg/kg body weight. A method of treatment may also include administering the active ingredient on a regimen of between one and four intakes per day.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy, for example, using methods such as those described in Gennaro et al. Remington's Pharmaceutical Sciences (18^{th} ed., Mack Publishing Company, 1990, see especially Part 8: Pharmaceutical preparations and their Manufacture). A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gellies, dressings, shampoos, tinctures, pastes, ointments, salves, powders and the like. Application of said compositions may be by aerosol, e.g. with a propellent such as nitrogen, carbon dioxide, a freon, or without a propellent such as a pump spray, drops, lotions, or a semisolid such as a thickened composition which can be applied by a swab. In particular, semisolid compositions such as salves, creams, gellies, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

In order to enhance the solubility and/or the stability of the compounds of formula (I) in pharmaceutical compositions, it can be advantageous to employ α-, β- or γ-cyclodextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Appropriate cyclodextrins are α-, β- or γ-cyclodextrins or ethers and mixed ethers thereof

wherein one or more of the hydroxy groups of the anhydroglucose units of the cyclodextrin are substituted with C₍₁₋₆₎alkyl, particularly methyl, ethyl or isopropyl, e.g. randomly methylated β-CD; hydroxy C₍₁₋₆₎alkyl, particularly hydroxyethyl, hydroxypropyl or hydroxybutyl; carboxy C₍₁₋₆₎alkyl, particularly carboxymethyl or carboxyethyl; C₍₁₋₆₎alkylcarbonyl, particularly acetyl; C₍₁₋₆₎alkyloxycarbonyl C₍₁₋₆₎alkyl or carboxy-C₍₁₋₆₎alkyloxy C₍₁₋₆₎alkyl, particularly carboxymethoxypropyl or carboxyethoxypropyl; C₍₁₋₆₎alkylcarbonyloxy C₍₁₋₆₎alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxypropyl-γ-CD and (2-carboxymethoxy)propyl-β-CD, and in particular 2-hydroxypropyl-β-CD (2-HP-β-CD).

The term mixed ether denotes cyclodextrin derivatives wherein at least two cyclodextrin hydroxy groups are etherified with different groups such as, for example, hydroxypropyl and hydroxyethyl.

The average molar substitution (M.S.) is used as a measure of the average number of moles of alkoxy units per mole of anhydroglucose. The M.S. value can be determined by various analytical techniques, preferably, as measured by mass spectrometry, the M.S. ranges from 0.125 to 10.

The average substitution degree (D.S.) refers to the average number of substituted hydroxyls per anhydroglucose unit. The D.S. value can be determined by various analytical techniques, preferably, as measured by mass spectrometry, the D.S. ranges from 0.125 to 3.

Experimental Part

Hereinafter, the term ‘RT’ means room temperature, ‘THF’ means tetrahydrofuran, ‘AcOH’ means CH₃COOH, ‘EtOH’ means ethanol, DME means dimethyl ether, DIPE means diisopropyl ether, iPrOH means isopropanol, DIAD means diisopropyl azodicarboxylate.

A. Preparation of the Intermediates

### EXAMPLE A1

a) Preparation of

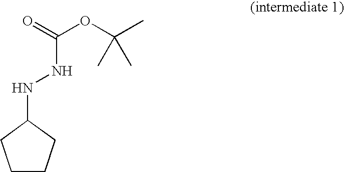

A mixture of tert-Butyl cyclopentylindenecarbazate (0.1 mol) and Pt/C₅% (2 g) in AcOH (30 ml) and CH₃OH (300 ml) was hydrogenated for 5 hours under a 3 bar pressure, then filtered over celite. The solvent was evaporated. The residue was taken up in ice water, basified with K2CO3 and extracted with CH2Cl2. The organic layer was separated, dried (MgSO4), filtered, and the solvent was evaporated. Yielding: 21 g of intermediate 1 (>100%).
b) Preparation of

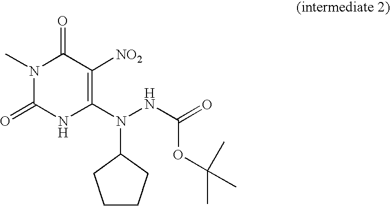

6-Chloro-3-methyl-5-nitro-2,4(1H,3H)-pyrimidinedione (0.038 mol) was added at room temperature to a mixture of intermediate 1 (0.047 mol) in CH₂Cl₂ (100 ml). The mixture was stirred for 4 hours. The solvent was evaporated. The residue was taken up in DIPE. The precipitate was filtered off and dried. Yielding: 13.5 g of intermediate 2 (96%).
c) Preparation of

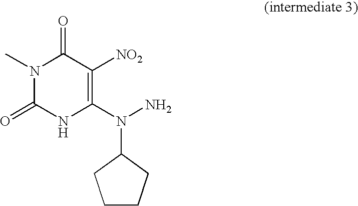

CF₃COOH (30 ml) was added at room temperature to a mixture of intermediate 2 (0.0365 mol) in CH₂Cl₂ (140 ml). The mixture was stirred at room temperature for 18 hours. The solvent was evaporated. The residue was crystallized from DIPE. The precipitate was filtered off and dried. Yielding: 8.55 g of intermediate 3 (61%).

### EXAMPLE A2

4a) Preparation of

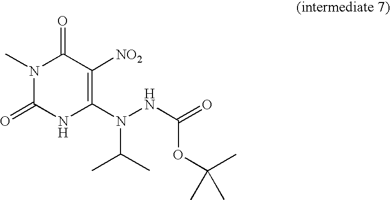

A mixture of 6-chloro-3-methyl-5-nitro-2,4(1H,3H)-pyrimidinedione (CA No.: 16689-35-3) (0.07 mol) and 2-(1-methylethyl)-1,1-dimethylethylester hydrazinecarboxylic acid (0.08 mol) in CH₂Cl₂ (180 ml) was stirred at room temperature for 18 hours. The solvent was evaporated. The residue was taken up in DIPE. The gum was decanted. Yielding: 32 g of intermediate 7. This product was used directly in the next reaction step.
b) Preparation of

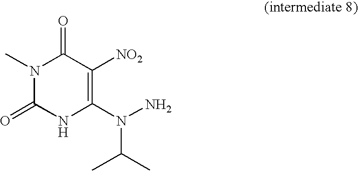

A mixture of intermediate 7 (0.07 mol) in CF₃COOH (55 ml) and CH₂Cl₂ (285 ml) was stirred at room temperature for 12 hours. The solvent was evaporated. The residue was taken up in DIPE. The gum was decanted. The residue was taken up in CH₂Cl₂. The solvent was evaporated. Yielding: 22 g of intermediate 8 (82%).
c) Preparation of 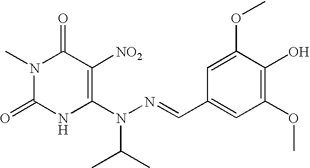
(intermediate 9)

NEt₃ (0.051 mol) then Tamis 3 Angstrom (4.3 g) then 2,6-dimethoxy-4-hydroxybenzaldehyde (0.0183 mol) were added to a mixture of intermediate 8 (0.0153 mol) in THF (170 ml). The mixture was stirred at 50° C. for 4 hours, then brought to room temperature and filtered. The filtrate was evaporated; The residue was taken up in CH₂Cl₂. The organic layer was washed with H₂O, dried (MgSO₄), filtered and the solvent was evaporated. Yielding: 6.6 g of intermediate 9 (>100%). This product was used without further purification.

### EXAMPLE A3

Preparation of

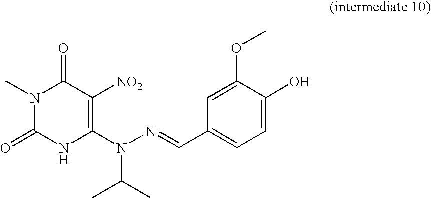

NEt₃ (0.0354 mol), Tamis 3 Angstrom (3 g) then vanillin (0.0129 mol) were added to a mixture of intermediate 8 (0.011 mol) in THF (120 ml). The mixture was stirred at 50° C. for 4 hours, then brought to room temperature and filtered. The filtrate was evaporated. The residue was taken up in H₂O. The mixture was extracted with CH₂Cl₂, then combined with intermediate 10. The organic layer was separated, dried (MgSO4), filtered, and the solvent was evaporated. Yielding: 5.1 g intermediate 10 (>100%).

### EXAMPLE A4

a) Preparation of

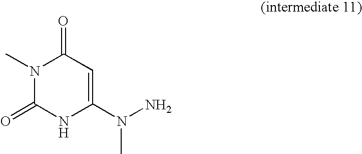

A mixture of 6-chloro-3-methyl-2,4(1H,3H)-pyrimidinedione (0.025 mol) and methylhydrazine (0.055 mol) in EtOH (25 ml) was stirred and refluxed for one hour and then was cooled in an ice water bath. The mixture was filtered to give a white solid. Yield: 3.4 g of intermediate 11.
b) Preparation of

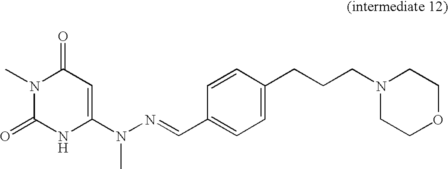

This experiment was performed twice. A mixture of intermediate 11 (0.01 mol) and 4-[2-(4-morpholinyl)ethoxy]-benzaldehyde (0.015 mol) in EtOH (30 ml) was stirred and refluxed for 3 hours then brought to room temperature. The precipitate was filtered off, rinsed with EtOH and dried. Yielding: 4.89 g of intermediate 12 (63%).

### EXAMPLE A5

a) Preparation of

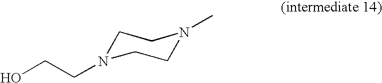

A mixture of N-methylpiperazine (0.0499 mol), 2-bromoethanol (0.0749 mol) and K₂CO₃ (0.0998 mol) in 2-butanone (90 mL) was stirred for 4 h at 90° C. The cooled reaction mixture was filtered. The filtrate was evaporated. Yielding 90% of intermediate 14. (Remark: lower yields were obtained on a higher scale and purification by short column chromatography was necessary).
b) Preparation of

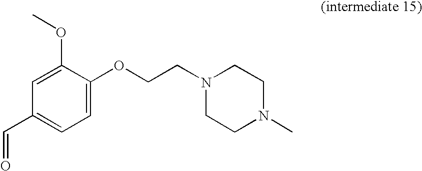

PPh₃ (0.0325 mol) was added dropwise at a temperature between 0 and 5° C. to a solution of Vanillin (CA No: 121-33-5) (0.025 mol), intermediate 14 (0.03 mol) and DIAD (0.0375 mol) in THF (60 ml). The mixture was stirred at room temperature for 18 hours. EtOAc was added. The mixture was extracted twice with HCl 3N. The acidic layer was washed with EtOAc, basified with K₂CO₃ and extracted with EtOAc. The organic layer was dried (MgSO₄), filtered, and the solvent was evaporated. Yielding: 3.9 g of intermediate 15 (56%).
c) Preparation of

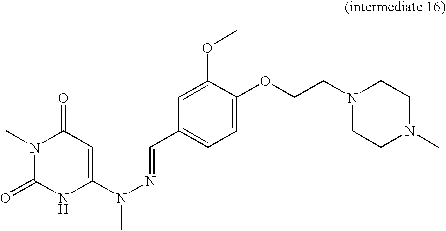

A mixture of intermediate 11 (0.011 mol) and intermediate 15 (0.014 mol) in EtOH (100 ml) was stirred and refluxed for 5 hours, then brought to room temperature and the solvent was evaporated. The residue was taken up in H₂O. The precipitate was filtered, washed with H₂O, then with DIPE. The precipitate was filtered off and dried. Yielding: 3.1 g of intermediate 16 (65%).

### EXAMPLE A6

a) Preparation of

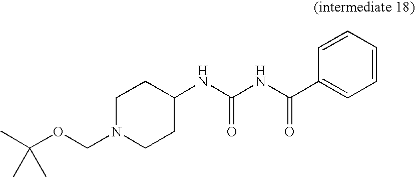

4-amino-1-Boc-piperidine (0.0484 mol) was added portionwise at 0° C. to a mixture of benzoylisocyanate (0.0533 mol) in CH₂Cl₂ (280 ml) under N₂ flow. The mixture was stirred at room temperature for 3 hours. The solvent was evaporated. The residue was crystallized from DIPE. The precipitate was filtered off and dried. Yielding: 7.75 g intermediate 18 (46%).
b) Preparation of

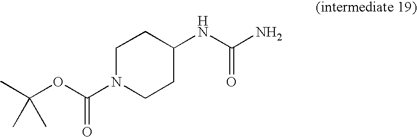

A mixture of intermediate 18 (0.0223 mol) and NaOH (0.38 mol) in CH₃OH (100 ml) and H₂O (100 ml) was stirred at room temperature for 12 hours, then stirred and refluxed for 1 hour and brought to room temperature. CH₃OH was evaporated. The precipitate was filtered, washed with H₂O and dried. Yielding: 4.46 g of intermediate 19 (82%).
c) Preparation of

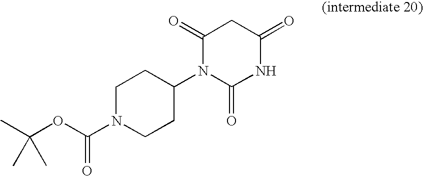

A mixture of intermediate 19 (0.0183 mol), diethylmalonate (0.02 mol) and EtONa/EtOH 21% (0.02 mol) in EtOH (60 ml) was stirred and refluxed for a week end, then brought to room temperature and the solvent was half-evaporated. The mixture was taken up in H₂O. HCl 3N was added til pH 5.5 was obtained. The mixture was extracted twice with CH₂Cl₂. The organic layer was separated, dried (MgSO₄), filtered, and the solvent was evaporated. The residue was taken up in cyclohexane. The precipitate was filtered off and dried. Yielding: 5.4 g of intermediate 20 (94%).
d) Preparation of

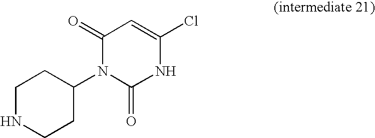

H₂O (0.0459 mol) was added dropwise slowly at room temperature to a mixture of intermediate 20 (0.017 mol) and POCl₃ (0.21 mol). The mixture was stirred and refluxed for 30 minutes, then brought to room temperature and the solvent was evaporated. The residue was taken up in ice. K₂CO₃ was added till pH 7 obtained. The mixture was washed with CH₂Cl₂ and the solvent was evaporated. The residue was taken up in DIPE. The precipitate was filtered off and dried. Yielding: 3.63 g of intermediate 21. This product was used without further purification.
e) Preparation of

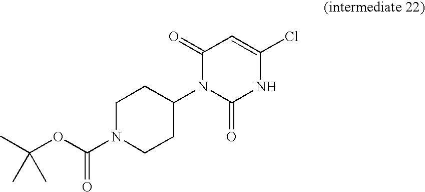

A mixture of intermediate 21 (0.017 mol) and di-tert-butyldicarbonate (0.026 mol) in CH₂Cl₂ (70 ml) and CH₃OH (15 ml) was stirred at room temperature for 12 hours. H₂O was added. The mixture was decanted. The solvent was evaporated. The residue was taken up in CH₂Cl₂. Activated carbon was added. The mixture was filtered over celite. The solvent was evaporated. The residue was taken up in DIPE. The precipitate was filtered off and dried. Yielding: 1.7 g of intermediate 22 (30%).
f) Preparation of

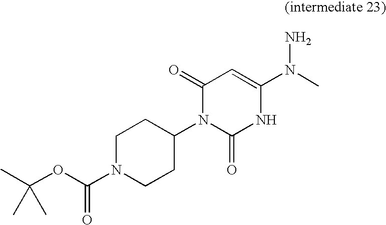

A mixture of intermediate 22 (0.0052 mol) and methylhydrazine (0.012 mol) in EtOH (20 ml) was stirred and refluxed for 1 hours, then brought to room temperature. The solvent was evaporated. Yielding: 1.76 g intermediate 23. This fraction was used without further purification.
g) Preparation of

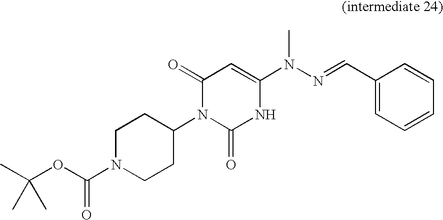

A mixture of intermediate 23 (0.0052 mol) and benzaldehyde (0.0065 mol) in EtOH (20 ml) was stirred and refluxed for 1 hour, then brought to room temperature and the solvent was evaporated. The residue was taken up in H₂O and extracted with CH₂Cl₂/CH₃OH. The organic layer was separated, dried (MgSO₄), filtered, and the solvent was evaporated. The residue (2.6 g) was purified by column chromatography over silica gel (eluent: CH₂Cl₂/CH₃OH 99.5/0.5; 15-40 μm). The pure fractions were collected and the solvent was evaporated. Yielding: 0.72 g of intermediate 24 (32%).

### EXAMPLE A7

Preparation of

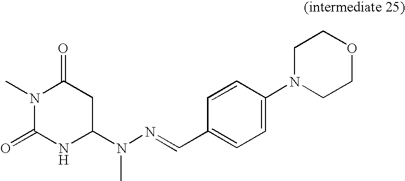

A mixture of intermediate 11 (0.0065 mol) and 4-morpholinobenzaldehyde (0.0071 mol) in EtOH (20 ml) was stirred and refluxed for 2 hours, then brought to room temperature. The precipitate was filtered off and dried. Yielding: 1.6 g of intermediate 25 (71%).

### EXAMPLE A8

a) Preparation of

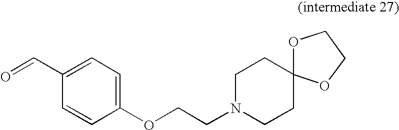

DIAD (0.0238 mol) was added dropwise at 5° C. to a solution of 4-hydroxybenzaldehyde (0.017 mol), 2-(4,4-ethylenedioxypiperidino)ethanol (CA No: 37443-73-5) (0.0204 mol) and P(Ph₃)₄ (0.0289 mol) in THF (60 ml). The mixture was stirred at 5° C. for 2 hours. H₂O (5 ml) was added. The mixture was extracted with HCl 3N, washed with EtOAc, basified with K₂CO₃ and extracted with EtOAc. The organic layer was separated, dried (MgSO₄), filtered, and the solvent was evaporated. Yielding: 7.6 g of intermediate 27.
b) Preparation of

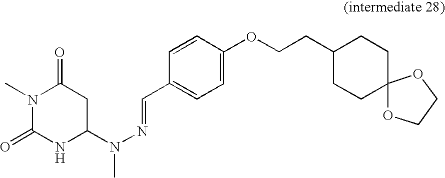

Intermediate 11 (0.018 mol) was added portionwise to a mixture of intermediate 27 (0.02 mol) in EtOH (130 ml). The mixture was stirred and refluxed for 2 hours and 30 minutes, then brought to room temperature and the solvent was evaporated. H₂O and CH₂Cl₂ were added. The organic layer was separated, dried (MgSO₄), filtered, and the solvent was evaporated. Yielding: 7.98 g of intermediate 28 (90%).

### EXAMPLE A9

Preparation of

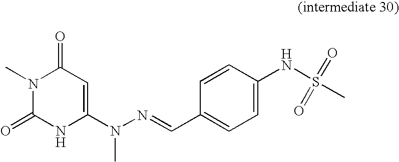

A mixture of intermediate 11 (0.0088 mol) and N-(4-formylphenyl)-methanesulfonamide (0.012 mol) in EtOH (20 ml) was stirred and refluxed for 3 hours, then brought to room temperature. The precipitate was filtered off and dried. Yielding: 2.34 g of intermediate 30 (75%).

### EXAMPLE A10

a) Preparation of 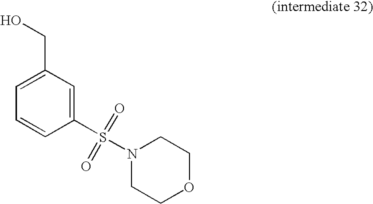
isobutylchloroformate (0.011 mol) then NEt₃ (0.0119 mol) were added dropwise at −15° C. to a mixture of 3-(4-morpholinylsulfonyl)-benzoic acid (0.0092 mol) in DME (30 ml) under N₂ flow. The mixture was stirred at 0° C. NaBH₄ (0.0184 mol) was added. The mixture was stirred at room temperature for 4 hours. H₂O was added dropwise. The mixture was acidified with HCl 3N and extracted twice with CH₂Cl₂. The organic layer was separated, dried (MgSO₄), filtered, and the solvent was evaporated. The residue was taken up in EtOAc. The precipitate was washed twice with K₂CO₃ 10%. The organic layer was separated, dried (MgSO₄), filtered, and the solvent was evaporated. The residue was purified by flash column chromatography over silica gel (eluent: CH₂Cl₂/CH₃OH 96/4; 70-200 μm). The pure fractions were collected and the solvent was evaporated. Yielding: 1.2 g of intermediate 32 (50%).
b) Preparation of

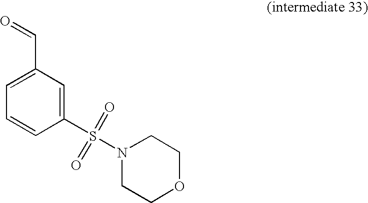

A solution of intermediate 32 (0.0047 mol) and DMSO (0.007 mol) in CH₂Cl₂ (5 ml) was added dropwise at −78° C. to a mixture of oxalyl chloride (0.0056 mol) and DMSO (0.007 mol) in CH₂Cl₂ (10 ml) under N₂ flow. The mixture was stirred for 30 minutes. NEt₃ (0.0235 mol) was added. The mixture was stirred at −78° C. for 5 minutes, then brought to room temperature. H₂O was added. The organic layer was separated, dried (MgSO4), filtered, and the solvent was evaporated. Yielding: 10.05 g of intermediate 33.
C) Preparation of

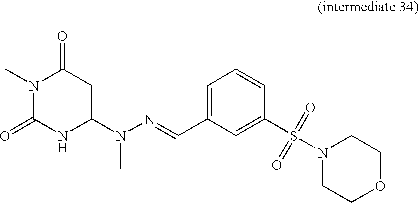

A mixture of intermediate 11 (0.0037 mol) and intermediate 33 (0.0041 mol) in EtOH (15 ml) was stirred and refluxed for 1 hour and 30 minutes, then brought to room temperature. The precipitate was filtered off and dried. Part of this fraction (0.17 g) was taken up in CH₃OH. The precipitate was filtered off and dried. Yielding: 0.11 g of intermediate 34.

### EXAMPLE A11

Preparation of

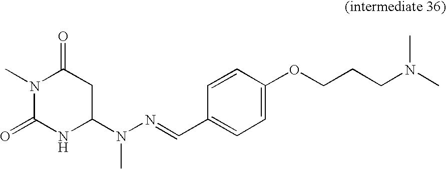

Intermediate 11 (0.004 mol) was added portionwise to a solution of 4-[3-(dimethylamino)propoxy]benzaldehyde (CA No: 26934-35-0) (0.0048 mol) in EtOH (25 ml). The mixture was stirred and refluxed for 4 hours, then stirred at room temperature for a week-end and three parts evaporated. The residue was diluted in DIPE. The precipitate was dried. Yielding: 1.3 g of intermediate 36 (90%).

### EXAMPLE A12

a) Preparation of

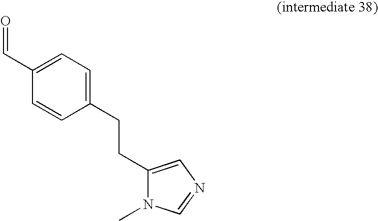

DIAD (0.0195 mol) was added dropwise at a temperature between 0 and 5° C. to a mixture of 4-hydroxybenzaldehyde (0.015 mol), 5-hydroxymethyl-1-methyl-1H-imidazole (CA No: 38993-84-9) (0.018 mol) and PPh₃ (0.0225 mol) in THF (40 ml) under N₂ flow. The mixture was stirred at room temperature overnight, then stirred for a week end, diluted in EtOAc, extracted with HCl 3N, washed with EtOAc, alkalinized with K₂CO₃ and extracted with EtOAc. The organic layer was separated, dried (MgSO₄), filtered, and the solvent was evaporated. Yielding: 1.6 g of intermediate 38 (49%).
b) Preparation of

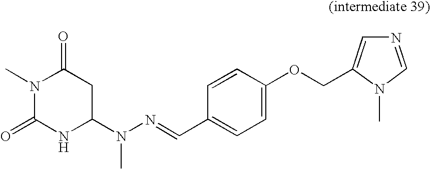

Intermediate 11 (0.0054 mol) was added portionwise to a solution of intermediate 38 (0.007 mol) in EtOH (30 ml). The mixture was stirred and refluxed for 2 hours, then cooled. The precipitate was filtered, washed with ethanol, then with diethyl ether and dried. The solvent was evaporated. The residue was taken up in H₂O. The mixture was filtered. The insoluble was taken up in ethanol. The solvent was evaporated till dryness. Yielding: 1.3 g of intermediate 39.

B. Preparation of the Compounds

### EXAMPLE B1

a) Preparation of

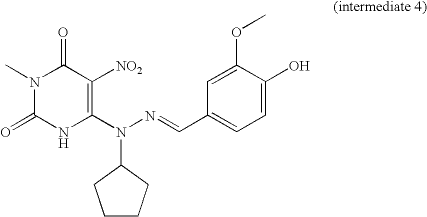

A mixture of intermediate 3 (0.0055 mol), vanillin (CA No.: 121-33-5) (0.0066 mol), Net₃ (0.0181 mol) and tamis 3 Angstrom (1.5 g) in THF (60 ml) was stirred at 50° C. for 3 hours, then brought to room temperature. The precipitate was filtered. The solvent was evaporated. The residue was taken up in H₂O. The mixture was extracted with CH₂Cl₂. The organic layer was separated, dried (MgSO4), filtered, and the solvent was evaporated. Yielding: 2 g of intermediate 4 (90%).
b) Preparation of

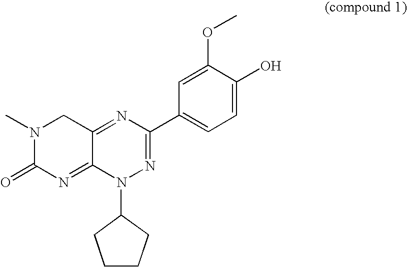

A mixture of intermediate 4 (0.005 mol) and Pd/C₅% (0.5 g) in EtOH (100 ml) was hydrogenated for 12 hours under a 1.5 bar pressure, then filtered over celite. Celite was washed with CH₂Cl₂/CH₃OH. The filtrate was evaporated. The residue was taken up in EtOH. The precipitate was filtered off and dried. Yielding: 0.3 g of compound 1 (16%).

### EXAMPLE B2

a) Preparation of

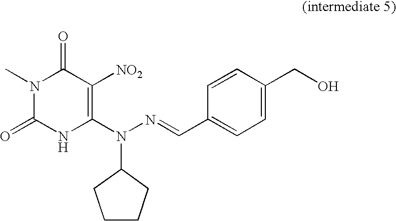

A mixture of intermediate 3 (0.028 mol), 4-(hydroxymethyl)-benzaldehyde (0.031 mol) and Net₃ (0.057 mol) in EtOH (280 ml) was stirred at 50° C. overnight. The solvent was evaporated. The residue was taken up in THF (200 ml). MgSO₄ (5 g) was added. The mixture was stirred at 50° C. for 2 hours, then brought to room temperature. The precipitate was filtered off and dried. Yielding: 15 g of intermediate 5 (>100%).
b) Preparation of

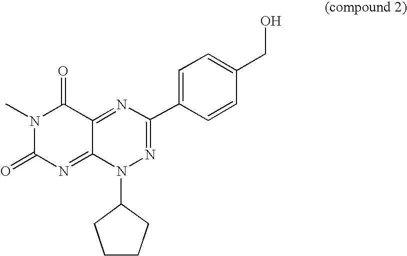

A mixture of intermediate 5 (0.028 mol) and Pd/C₅% (3 g) in EtOH (300 ml) was hydrogenated at room temperature for 12 hours, then filtered over celite. Celite was washed with CH₂Cl₂/CH₃OH. The filtrate was evaporated. The residue was taken up in H₂O. The mixture was taken up in CH₂Cl₂/CH₃OH. The organic layer was separated, dried (MgSO₄), filtered, and the solvent was evaporated. The residue (9 g) was purified by column chromatography over silica gel (eluent: CH₂Cl₂/CH₃OH 97/3; 20-45 μm). The pure fractions were collected and the solvent was evaporated. Yielding: 0.32 g of compound 2 (3.2%).
c) Preparation of

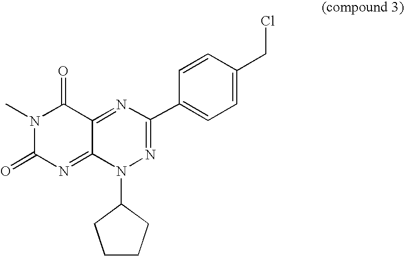

A mixture of compound 2 (0.0009 mol) and SOCl₂ (0.0036 mol) in CH₂Cl, (30 ml) was stirred at room temperature for 12 hours. The solvent was evaporated. Yielding: 0.34 g of compound 3.

### EXAMPLE B3

a) Preparation of

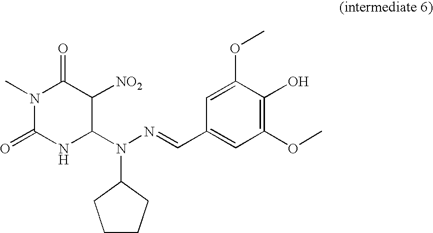

A mixture of intermediate 3 (0.0055 mol), 2,6-dimethoxy-4-hydroxybenzaldehyde (0.0066 mol) and NEt₃ (0.018 mol) in tamis 3 Angstrom (1.5 ml) and THF (60 ml) was stirred at 50° C. for 3 hours. The precipitate was filtered. The filtrate was evaproated. The residue was taken up in H₂O/CH₂Cl₂. The mixture was filtered. The organic layer was separated, dried (MgSO₄), filtered, and the solvent was evaporated. Yielding: 2.4 g of intermediate 6. This product was used directly in the next reaction step.
b) Preparation of

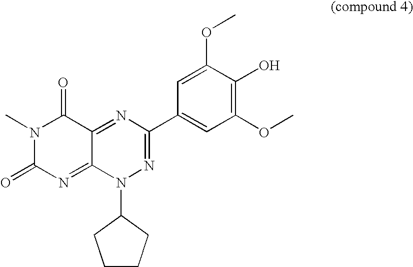

A mixture of intermediate 6 (0.0051 mol) and Pd/C₅% (0.5 g) in EtOH (100 ml) was hydrogenated for 16 hours under a 1.5 bar pressure, then filtered over celite. Celite was washed with CH₂Cl₂/CH₃OH. The filtrate was evaporated. The residue was taken up in EtOH. The precipitate was filtered off and dried. Yielding: 0.25 g of compound 4 (12%) which could be further modified as for example provided in examples B5, B19.

### EXAMPLE B4

a) Preparation of

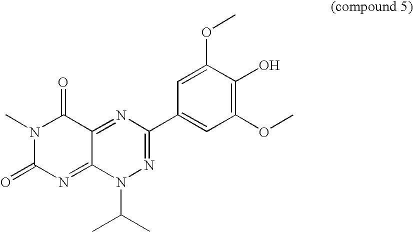

A mixture of intermediate 9 (0.0153 mol) and Pd/C 10% (1 g) in EtOH (200 ml) was hydrogenated at room temperature for 16 hours under a 1.5 bar pressure, then filtered over celite. Celite was washed with CH₂Cl₂/CH₃OH. The filtrate was evaporated. The residue was taken up in iPrOH. The precipitate was filtered, washed with iPrOH, then with DIPE and dried. Yielding: 0.5 g of compound 5 which could be further modified as for example provided in examples B5, B19.

### EXAMPLE B5

a) Preparation of

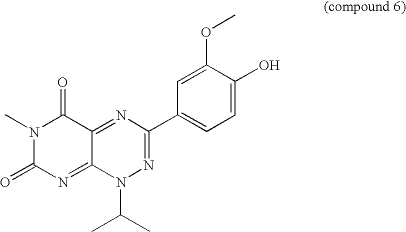

A mixture of intermediate 10 (0.0136 mol) and Pd/C₅% (1 g) in EtOH (200 ml) was hydrogenated at room temperature for 18 hours under a 1.5 bar pressure, then filtered over celite. Celite was washed with CH₂Cl₂/CH₃OH. The filtrate was evaporated. The residue was taken up in iPrOH. The precipitate was filtered, washed with iPrOH, then with DIPE and dried (0.17 g, 3.6%). Celite was washed again with CH₂Cl₂/CH₃OH. The precipitate was filtered off and dried. Yielding: 0.12 g of compound 6 (6.2%).
b) Preparation of

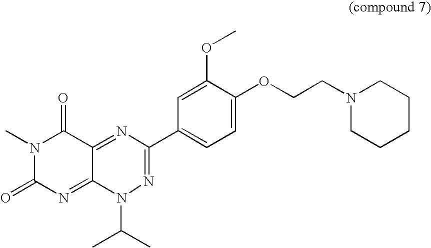

DIAD (0.0013 mol) was added dropwise at 0° C. to a solution of compound 6 (0.0008 mol), N-piperidine-ethanal (CA No.: 3040-44-6) (0.0012 mol) and PPh₃ (0.0013 mol) in THF (12 ml) under N₂ flow. The mixture was stirred at room temperature for 12 hours. H₂O was added. The mixture was extracted twice with CH₂Cl₂. The organic layer was separated, dried (MgSO₄), filtered, and the solvent was evaporated. The residue (1.45 g) was purified by column chromatography over silica gel (eluent: CH₂Cl₂/CH₃OH 88/12; 15-40 μm). The pure fractions were collected and the solvent was evaporated. The residue (0.2 g) was taken up in DIPE. The precipitate was filtered off and dried. Yielding: 0.17 g of compound 7 (44%).

### EXAMPLE B6

a) Preparation of

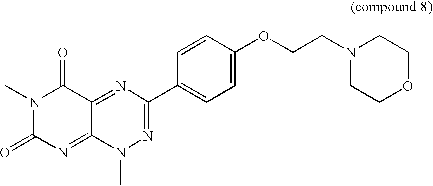

NaNO₂ (0.019 mol) was added at 5° C. to a mixture of intermediate 12 (0.0125 mol) in H₂O (3.1 ml) and AcOH (50 ml). The mixture was stirred at 5° C. for 30 minutes. DIPE was added. The residue was taken up in CH₂Cl₂/K₂CO₃ 10%. The mixture was stirred for 15 minutes and filtered over celite. The celite was rinsed with CH₂Cl₂. The organic layer was separated, dried (MgSO₄), filtered, and the solvent was evaporated. Yielding: 2.4 g of compound 8 and its nitrosoderivative 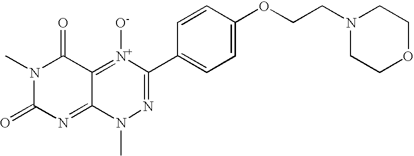
b) Preparation of

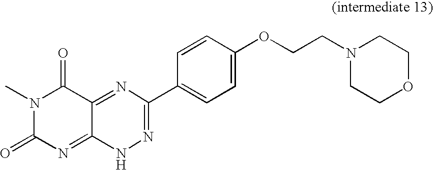

A mixture of compound 8 (0.0030 mol) and its nitrosoderivative (0.0030 mol) in DMF (20 ml) was stirred at 90° C. for 2 hours then brought to room temperature, poured out into ice water. The precipitate was filtered off and dried. Yielding: 1.34 g of intermediate 13 (59%).
c) Preparation of

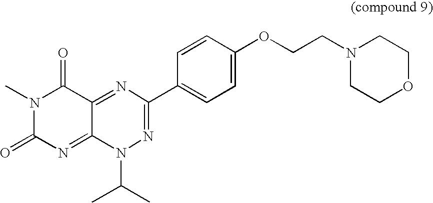

A mixture of intermediate 13 (0.0044 mol), 2-iodopropane (0.02 mol) and K₂CO₃ (0.0131 mol) in dioxane (200 ml) was stirred and refluxed for 12 hours, then brought to room temperature. The solvent was evaporated. The residue was taken up in H₂O. The mixture was filtered, washed with H₂O, then with EtOH, then with DIPE and dried. The residue (0.85 g) was taken up in EtOH. The precipitate was filtered off and dried. Yielding: 0.682 g of compound 9 (36%).

### EXAMPLE B7

a) Preparation of

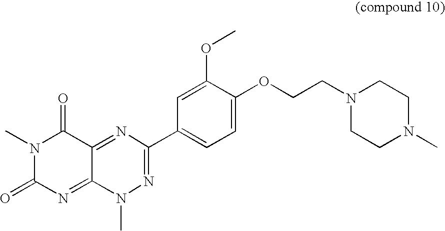

NaNO₂ (0.011 mol) was added at a temperature between 0 and 5° C. to a mixture of intermediate 16 (0.0072 mol) in H₂O (1.75 ml) and AcOH (27 ml). The mixture was stirred at 10° C. for 2 hours, then diluted in DIPE. The precipitate was filtered off and dried. Yielding: 5 g of compound 10 and its nitrosoderivative (>100%).
b) Preparation of

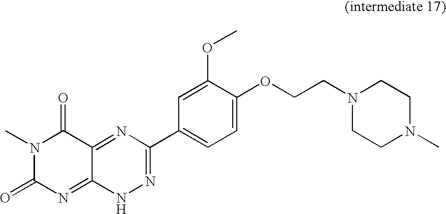

A mixture of compound 17 (0.0038 mol) and its nitrosoderivative (0.0038 mol) in DMF (22 ml) was stirred at 100° C. for 1 hour, then brought to room temperature and diluted in DIPE. The precipitate was filtered off and dried. Yielding: 2.9 g of intermediate 17 (94%).
c) Preparation of

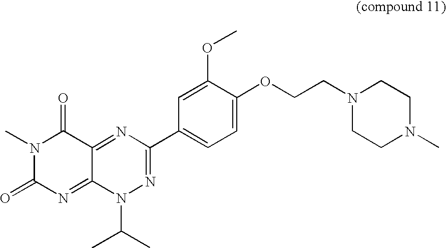

A mixture of intermediate 17 (0.0033 mol), 2-iodopropane (0.015 mol) and K₂CO₃ (0.0098 mol) in dioxane (150 ml) was stirred and refluxed for 16 hours, then brought to room temperature and the solvent was evaporated. The residue was taken up in H₂O. The mixture was extracted with CH₂Cl₂. The organic layer was separated, dried (MgSO₄), filtered, and the solvent was evaporated. The residue was taken up in EtOH. The precipitate was filtered off and dried. This fraction was dried at 80° C. for 3 hours under a vacuo. Yielding: 0.411 g of compound 11 (26%).

### EXAMPLE B8

Preparation of

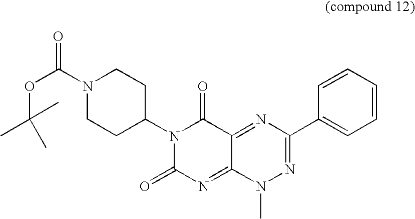

NaNO₂ (0.0022 mol) was added at 5° C. to a mixture of intermediate 24 (0.0015 mol) in AcOH (6 ml) and H₂O (0.6 ml). The mixture was brought to room temperature, then stirred for 6 hours. Diethyl ether was added. The precipitate was filtered off and dried. The residue (0.67 g) was purified by column chromatography over silica gel (eluent: CH₂Cl₂/CH₃OH 99/1; 15-40 μm). The pure fractions were collected and the solvent was evaporated. Yielding: 0.3 g of compound 12 (45%).

### EXAMPLE B9

a) Preparation of

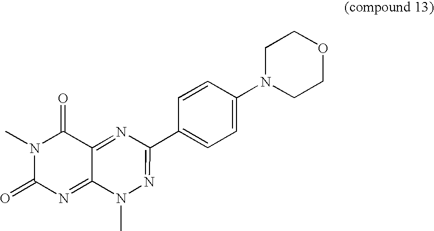

NaNO₂ (0.0125 mol) was added portionwise at 5° C. to a mixture of intermediate 25 (0.0104 mol) in CH₃COOH (35 ml) and H₂O (1.8 ml). The mixture was stirred at 5° C. for 30 minutes. Ethylic ether was added. The precipitate was filtered off and dried. Yielding: 3.85 g compound 13 and its nitrosoderivative (quantitative).
b) Preparation of

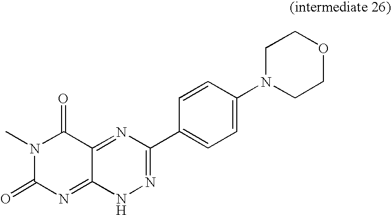

A mixture of compound 13 (0.0052 mol) and its nitrosoderivative (0.0052 mol) in DMF (38 ml) was stirred at 90° C. for 3 hours and poured out into H₂O. The precipitate was filtered off and dried. Yielding: 2.03 g of intermediate 26 (57%) which can be further modified for example as described in examples B14-B18.

### EXAMPLE B10

a) Preparation of

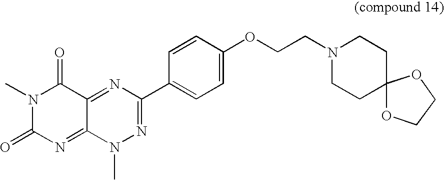

NaNO₂ (0.0176 mol) was added portionwise at a temperature between 5 and 10° C. to a solution of intermediate 28 (0.016 mol) in AcOH (37.3 ml) and H₂O (2 ml). The mixture was stirred at 10° C. for 2 hours, poured out into DIPE. The precipitate was filtered. The mixture was taken up in CH₂Cl₂/CH₃OH. The solvent was evaporated. Yielding: 7.3 g of compound 14 (100%).
b) Preparation of

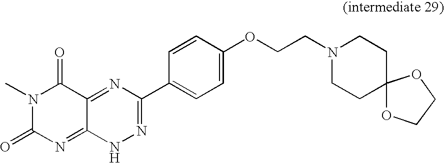

A mixture of compound 14 (0.0073 mol) and its nitrosoderivative (0.0073 mol) in DMF (30 ml) was stirred at 90° C. for 4 hours. The precipitate was filtered. The filtrate was evaporated. Yielding: intermediate 29 (22%) which can be further modified to compounds of formula 1, for example as described in examples B14-B18.

### EXAMPLE B11

a) Preparation of

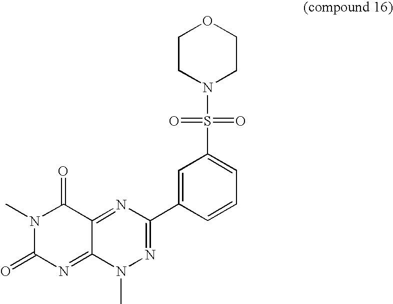

NaNO₂ (0.0029 mol) was added at 5° C. to a mixture of intermediate 34 (0.0022 mol) in H₂O (0.55 ml) and AcOH (15 ml). The mixture was stirred at room temperature for 48 hours, poured out on ice and basified with K₂CO₃. The precipitate was filtered, washed with iPrOH and dried. Yielding: 0.9 g compound 16 and its nitrosoderivative (100%). This product was used without further purification.
b) Preparation of

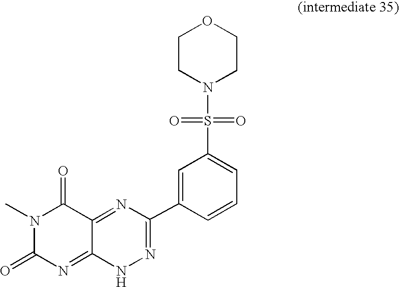

A mixture of compound 16 (0.0010 mol), its nitrosoderivative (0.0010 mol) and 1,4-dimercapto-2,3-Butanediol (0.0064 mol) in CH₃OH (10 ml) was stirred at room temperature for 3 days. 1,4-dimercapto-2,3-Butanediol (0.0064 mol) was added. The mixture was stirred for 1 day more, poured out into H₂O, extracted with CH₂Cl₂ and filtered. Yielding: 0.2 g of intermediate 35. The organic layer was separated, dried (MgSO₄), filtered and the solvent was evaporated. The residue was purified by column chromatography over kromasil (eluent: CH₂Cl₂/EtOAc 95/5; 5 μm). The pure fractions were collected and the solvent was evaporated. Yielding: 0.084 g of compound 16 (10%). Intermediate 35 may be further modified to compounds of formula I, such as provided in examples B14-B18.

### EXAMPLE B12

a) Preparation of

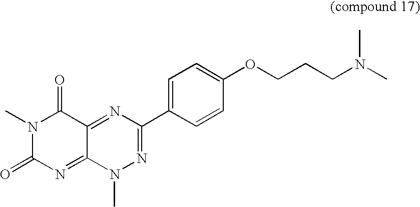

NaNO₂ (0.0041 mol) was added portionwise at a temperature between 0 and 5° C. to a mixture of intermediate 36 (0.0036 mol) in AcOH (15 ml) and H₂O (0.8 ml). The mixture was stirred at 10° C. for 3 hours, then stirred at room temperature overnight and diluted in DIPE. The gum was taken up in CH₂Cl₂/CH₃OH and evaporated till dryness. Yielding: 2 g of compound 17 and its nitrosoderivative (mixture). This mixture was used directly in the next reaction step.
b) Preparation of

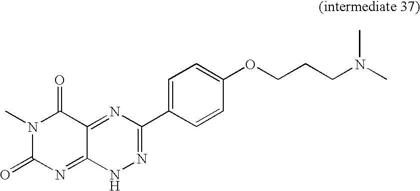

A mixture of compound 17 (0.0018 mol) and its nitrosoderivative (0.0018 mol) in DMF (15 ml) was stirred at 90° C. for 4 hours, then cooled, washed with DIPE and dried. Yielding: intermediate 37 (47%) which could be converted in compounds of formula I, for example as described in examples B14-B18.

### EXAMPLE B13

a) Preparation of

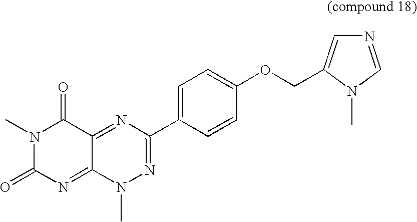

A mixture of intermediate 39 (0.0035 mol) in AcOH (15 ml) and H₂O (0.8 ml) was cooled to a temperature between 0 and 5° C. NaNO₂ (0.004 mol) was added portionwise. The mixture was stirred at 10° C. for 3 hours. DIPE (250 ml) was added. The precipitate was filtered, washed with DIPE and dried. Yielding: 1 g of compound 18 and its nitrosoderivative (mixture).
b) Preparation of

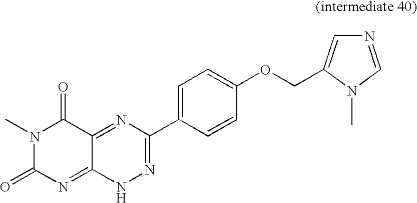

A mixture of compound 18 (0.0013 mol) and its nitrosoderivative (0.0013 mol) in DMF (10 ml) was stirred at 90° C. for 4 hours, then cooled and the solvent was evaporated in vacuo. The precipitate was filtered, washed with diethyl ether and dried. Yielding: 0.9 g of intermediate 40. This product was used directly in the next reaction step, to convert it into a compound of formula I, using amongst others the reaction schemes as provided in examples B15-B19.

### EXAMPLE B14

Preparation of

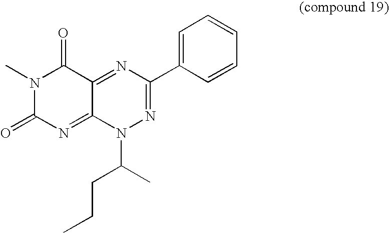

K₂CO₃ (0.0068 mol) then 2-bromopentane (0.0117 mol) were added to a mixture of 6-methyl-3-phenyl-pyrimido[5,4-e]-1,2,4-triazine-5,7(1H, 6H)-dione (CA No.: 42285-76-7) (0.0039 mol) in dioxane (60 ml). The mixture was stirred and refluxed for 48 hours. The solvent was evaporated till dryness. The residue was taken up in CH₂Cl₂ and washed with H₂O. The organic layer was separated, dried (MgSO₄), filtered and the solvent was evaporated. The residue (1 g) was purified by column chromatography over silica gel (eluent: CH₂Cl₂/CH₃OH 99.5/0.5; 35-70 cm). The pure fractions were collected and the solvent was evaporated. The residue (0.45 g) was crystallized from EtOH. The precipitate was filtered off and dried. Yielding: 0.15 g of compound 19 (12%).

### EXAMPLE B15

Preparation of

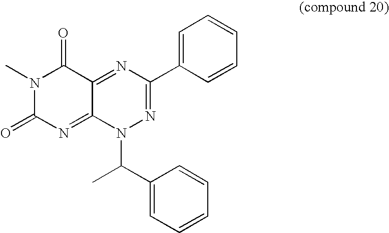

K₂CO₃ (0.0034 mol) then (1-bromoethyl)benzene (0.0058 mol) were added to a mixture of 6-methyl-3-phenyl-pyrimido[5,4-e]-1,2,4-triazine-5,7(1H, 6H)-dione (CA No.: 42285-76-7) hereinafter referred to as intermediate 41 (0.0019 mol) in dioxane (45 ml). The mixture was stirred and refluxed for 5 hours. The solvent was evaporated til dryness. The residue was taken up in H₂O and extracted with CH₂Cl₂. The organic layer was separated, dried (MgSO4), filtered, and the solvent was evaporated. The residue was purified by column chromatography over silica gel (eluent: CH₂Cl₂/CH₃OH 99/1; 35-70 μm). The pure fractions were collected and the solvent was evaporated. The residue was crystallized from 2-propanol. The precipitate was filtered off and dried. Yielding: 0.18 g of compound 20 (26%).

### EXAMPLE B16

Preparation of

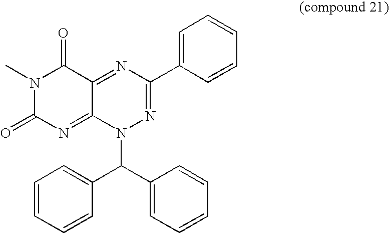

A mixture of intermediate 41 (0.0075 mol), bromodiphenylmethane (0.0082 mol) and K₂CO₃ (0.0082 mol) in dioxane (70 ml) was stirred and refluxed for 1 hour, then brought to room temperature and the solvent was evaporated. The residue was taken up in H₂O and extracted twice with CH₂Cl₂. The organic layer was separated, dried (MgSO₄), filtered, and the solvent was evaporated. The residue was taken up in EtOH. The precipitate was filtered off and dried. Yielding: 0.213 g of compound 21.

### EXAMPLE B 17

Preparation of

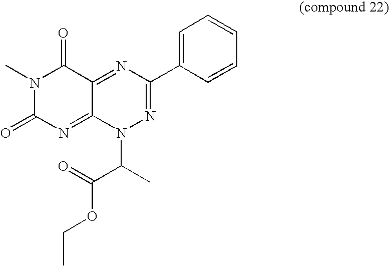

A mixture of intermediate 41 (0.0039 mol), ethyl-2-bromopropionate (CA No.: 535-11-5) (0.0117 mol) and K₂CO₃ (0.0117 mol) in dioxane (50 ml) was stirred at 100° C. for 1 hour. The solvent was evaporated. The residue was taken up in CH₂Cl₂. The organic layer was washed with H₂O, separated, dried (MgSO₄), filtered and the solvent was evaporated. The residue (1.2 g) was purified by column chromatography over silica gel (eluent: CH₂Cl₂/CH₃OH 99.5/0.5; 15-40 μm). The pure fractions were collected and the solvent was evaporated. The residue was crystallized from diethyl ether. The precipitate was filtered off and dried. Yielding: 0.06 g of compound 22 (4%).

### EXAMPLE B18

a) Preparation of

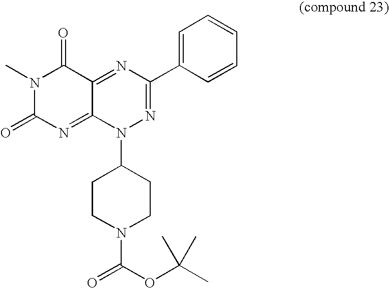

A mixture of intermediate 41 (0.0078 mol), tert-butyl-4-iodopiperidine-1-carboxylate (CA No.: 301673-14-3) (0.0235 mol) and K₂CO₃ (2.17 g) in dioxane (150 ml) was stirred and refluxed in a sealed vessel overnight. The solvent was evaporated till dryness. The residue was taken up in H₂O. The mixture was extracted with CH₂Cl₂. The organic layer was separated, dried (MgSO₄), filtered, and the solvent was evaporated. The residue was crystallized from EtOH. Yielding: 0.95 g compound 23 (28%).
b) Preparation of

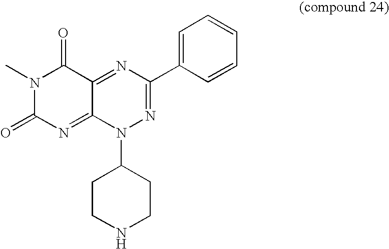

A mixture of compound 24 (0.0005 mol) in Hcl (5-6N in isopropanol) (0.4 ml) and isopropanol (10 ml) was stirred at 50° C. for a week end. The precipitate was filtered off and dried. Yielding: 0.15 g of compound 24 (84%).

### EXAMPLE B19

a) Preparation of

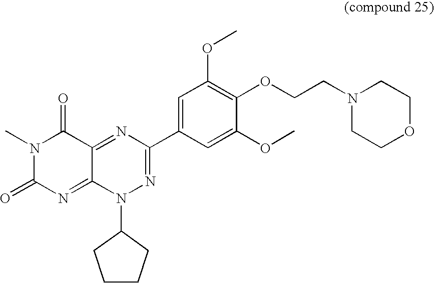

DIAD (0.0008 mol) was added at 5° C. to a mixture of compound 4 (0.0006 mol), N-piperidine-ethanol (CA No.: 3040-44-6) (0.0007 mol) and PPh₃ (0.0009 mol) in THF (5 ml) under N₂ flow. The mixture was stirred at room temperature for 12 hours, poured out into H₂O and extracted with CH₂Cl₂. The organic layer was separated, dried (MgSO₄), filtered, and the solvent was evaporated. The residue (1 g) was purified by column chromatography over silica gel (eluent: CH₂Cl₂/CH₃OH 97/3; 15-35 μm). The pure fractions were collected and the solvent was evaporated. The residue (0.09 g) was taken up in DIPE. The precipitate was filtered off and dried. Yielding: 0.058 g of compound 25 (18%).

Tables 1 & 2 list compounds of the present invention as prepared according to one of the above examples.

TABLE 1

| | |
|---|---|
| | (I) |
| 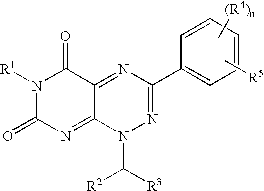 | |

| Co. | | | | | | | Physical |
|---|---|---|---|---|---|---|---|
| No. | R¹ | R² | R³ | n | R⁴ | R⁵ | data |
| 15 | CH₃ | H | H | 2 | 3,4-Cl | — | — |
| 26 | CH₃ | H | CH₃ | 2 | 3,4-Cl | — | mp |
| | | | | | | | 191.9- |
| | | | | | | | 295.6° C. |
| 27 | CH₃ | H | CH₃ | 0 | — | 4 methoxy | — |
| 28 | CH₃ | H | CH₃ | 1 | 3 | 4 methoxy | — |
| | | | | | methoxy | | |
| 29 | CH₃ | H | H | 0 | — | — | — |
| 30 | CH₃ | H | H | 0 | — | 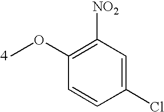 | — |
| 31 | CH₃ | H | H | 0 | — | 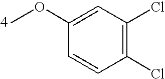 | — |
| 32 | CH₃ | H | H | 0 | — | 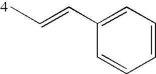 | — |
| 33 | CH₃ | H | H | 0 | — | 4 phenyl | — |
| 34 | CH₃ | C₂H₄—OH | H | 0 | — | 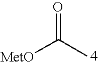 | mp >250° C. |
| 35 | CH₃ | CH₃ | H | 2 | 3,5 | — | mp > |
| | | | | | methoxy | | 250° C. |
| 36 | CH₃ | CH₃ | H | 1 | 3 F | 4 methoxy | — |
| 37 | CH₃ | CH₃ | H | 2 | 3,6 | 4 methoxy | — |
| | | | | | methyl | | |
| 38 | CH₃ | CH₃ | H | 0 | — | 3-trifluoromethyl | — |
| 39 | CH₃ | CH₃ | H | 0 | — | 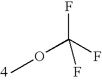 | — |
| 40 | CH₃ | CH₃ | H | 1 | 3 F | 4 methoxy | — |
| 41 | CH₃ | CH₃ | H | 0 | — | 4-O—C₄H₉ | — |
| 42 | CH₃ | CH₃ | H | 0 | — | 4-CN | — |
| 43 | CH₃ | C₂H₄—OH | H | 2 | 3,5-Cl | — | — |
| 44 | CH₃ | CH₃ | H | 1 | 3 | — | mp > |
| | | | | | methoxy | | 250° C. |
| 45 | CH₃ | CH₃ | H | 0 | — | 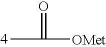 | mp >250° C. |
| 46 | CH₃ | CH₃ | H | 0 | — | 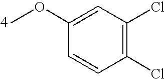 | — |
| 47 | CH₃ | CH₃ | H | 0 | — | 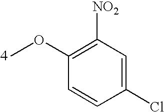 | — |
| 48 | CH₃ | CH₃ | H | 2 | 3,5-Cl | — | — |
| 49 | CH₃ | C₂H₄—OH | H | 1 | 4-Cl | — | — |
| 50 | CH₃ | C₂H₄—OH | H | 1 | 3 | — | — |
| | | | | | methoxy | | |
| 51 | CH₃ | H | phenyl | 2 | 3,5 | — | — |
| | | | | | methoxy | | |
| 52 | 2-propanyl | CH₃ | H | 2 | 3,5 | — | — |
| | | | | | methoxy | — | — |
| 53 | CH₃ | CH₃ | CH₃ | 0 | — | — | mp > |
| | | | | | | | 250° C. |
| 54 | CH₃ | CH₃ | CH₃ | 0 | — | 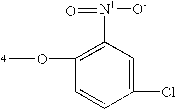 | — |
| 55 | CH₃ | CH₃ | C₂H₅ | 2 | 3,5 | H | — |
| | | | | | methoxy | | |
| 56 | CH₃ | CH₃ | CH₂—C₆H₅ | 2 | 3,5 | H | — |
| | | | | | methoxy | | |
| 57 | CH₃ | CH₃ | CH₃ | 2 | 3,5 | H | — |
| | | | | | methoxy | | |
| 58 | 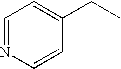 | H | H | 0 | — | — | — |
| 59 | 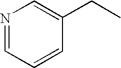 | H | H | 0 | — | — | mp 314.7-321.5° C. |
| 60 | phenyl | H | H | 0 | — | — | — |
| 61 | 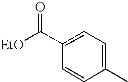 | H | H | 0 | — | — | — |
| 62 | 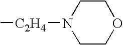 | H | H | 0 | — | — | — |
| 63 | 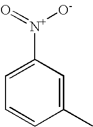 | H | H | 0 | — | — | — |
| 64 | naphtyl | H | H | 0 | — | — | — |
| 67 | CH₃ | H | 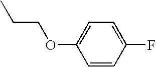 | 0 | — | — | — |
| 19 | CH₃ | CH₃ | C₂H₄—CH₃ | 0 | — | — | mp 216° C. |
| 20 | CH₃ | CH₃ | C₆H₅ | 0 | — | — | — |
| 21 | CH₃ | C₆H₅ | C₆H₅ | 0 | — | — | mp > 250 ° C. |
| 12 | 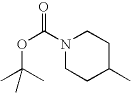 | H | H | 0 | — | — | mp > 250° C. |
| 68 | CH₃ | CH₃ | CH₂—C₆H₅ | 0 | — | — | mp 216° C. |
| 16 | CH₃ | H | H | 0 | — | 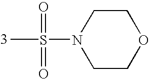 | — |
| 22 | CH₃ | CH₃ | 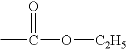 | 0 | — | — | mp 215° C. |
| 8 | CH₃ | H | H | 0 | — | 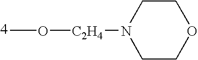 | — |
| 69 | CH₃ | CH(CH₃)₂ | C₆H₅ | 0 | — | — | mp 239° C. |
| 13 | CH₃ | H | H | 0 | — | 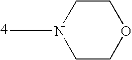 | — |
| 14 | CH₃ | H | H | 0 | — | 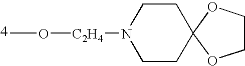 | — |
| 70 | CH₃ | H | H | 0 | — | 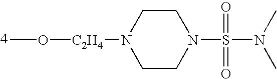 | — |
| 9 | CH₃ | CH₃ | CH₃ | 0 | — | 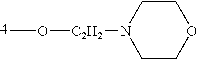 | mp 232° C. |
| 71 | CH₃ | CH₃ | CH₃ | 1 | 3 methoxy | 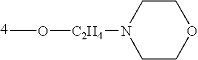 | mp 240° C. |
| 10 | CH₃ | H | H | 1 | 3 methoxy | 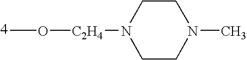 | mp 239° C. |
| 11 | CH₃ | CH₃ | CH₃ | 1 | 3 methoxy | 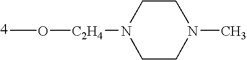 | mp 233° C. |
| 6 | CH₃ | CH₃ | CH₃ | 1 | 3 | 4-OH | — |
| | | | | | methoxy | | |
| 7 | CH₃ | CH₃ | CH₃ | 1 | 3 methoxy | 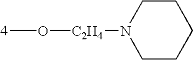 | mp 222° C. |
| 72 | CH₃ | CH₃ | CH₃ | 0 | — | 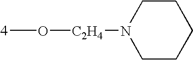 | mp 225° C. |
| 73 | CH₃ | CH₃ | CH₃ | 0 | — | 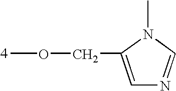 | — |
| 74 | CH₃ | CH₃ | CH₃ | 1 | 3 Cl | 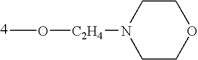 | — |
| 18 | CH₃ | H | H | 0 | — | 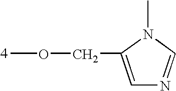 | — |
| 5 | CH₃ | CH₃ | CH₃ | 2 | 3,5 | 4-OH | — |
| | | | | | methoxy | | |
| 75 | CH₃ | CH₃ | CH₃ | 2 | 3,5 methoxy | 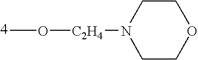 | — |
| 17 | CH₃ | H | H | 0 | — | 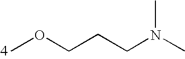 | — |
| 76 | CH₃ | CH₃ | CH₃ | 0 | — | 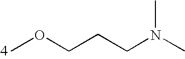 | — |
| 77 | CH₃ | CH₃ | CH₃ | 2 | 3,5 methoxy | 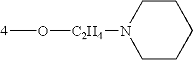 | — |
| 78 | CH₃ | CH₃ | CH₃ | 1 | 3 methoxy | 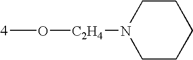 | — |
| 79 | CH₃ | CH₃ | CH₃ | 0 | — | 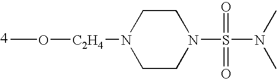 | — |
| 80 | CH₃ | CH₃ | CH₃ | 0 | — | 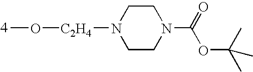 | — |
| 81 | CH₃ | CH₃ | CH₃ | 0 | — | 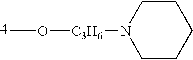 | — |
| 82 | CH₃ | CH₃ | CH₃ | 0 | — | 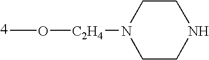 | — |
| 83 | CH₃ | CH₃ | CH₃ | 0 | — | 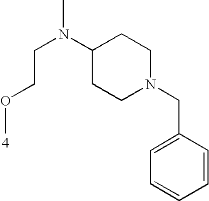 | — |
| 84 | CH₃ | CH₃ | CH₃ | 0 | — | 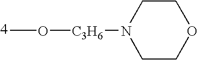 | — |
| 85 | CH₃ | CH₃ | CH₃ | 2 | 3,5 methoxy | 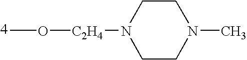 | — |
| 86 | CH₃ | CH₃ | CH₃ | 2 | 3,5 methoxy | 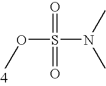 | — |
| 87 | CH₃ | CH₃ | CH₃ | 0 | — | 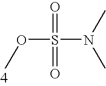 | — |
| 88 | CH₃ | CH₃ | CH₃ | 1 | 3 Cl | 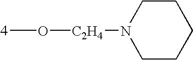 | — |
| 89 | CH₃ | CH₃ | CH₃ | 2 | 3,5 methoxy | 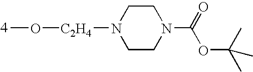 | — |
| 90 | CH₃ | CH₃ | CH₃ | 0 | — | 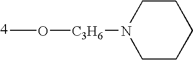 | — |
| 91 | CH₃ | CH₃ | CH₃ | 0 | — | 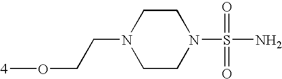 | — |
| 92 | CH₃ | CH₃ | CH₃ | 1 | 3 Cl | 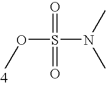 | — |
| 93 | CH₃ | CH₃ | CH₃ | 0 | — | 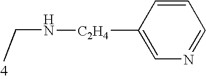 | — |
| 94 | CH₃ | CH₃ | CH₃ | 0 | — | 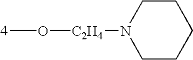 | — |
| 95 | CH₃ | CH₃ | CH₃ | 0 | — | 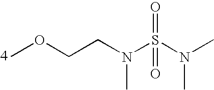 | — |
| 96 | CH₃ | CH₃ | CH₃ | 2 | 3,5 methoxy | 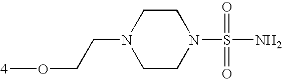 | — |
| 97 | CH₃ | CH₃ | CH₃ | 0 | — | 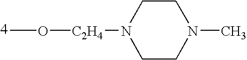 | — |
| 98 | CH₃ | CH₃ | CH₃ | 0 | — | 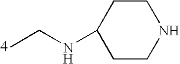 | — |
| 99 | CH₃ | CH₃ | CH₃ | 0 | — | 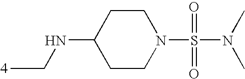 | — |
| 100 | CH₃ | CH₃ | CH₃ | 0 | — | 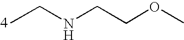 | — |
| 101 | CH₃ | CH₃ | CH₃ | 0 | — | 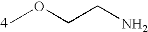 | — |
| 102 | CH₃ | CH₃ | CH₃ | 0 | — | 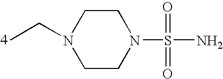 | — |
| 103 | CH₃ | CH₃ | CH₃ | 0 | — | 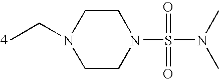 | — |
| 104 | CH₃ | CH₃ | CH₃ | 1 | 3 Cl | 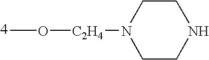 | — |
| 105 | CH₃ | CH₃ | CH₃ | 1 | 3,5 methoxy | 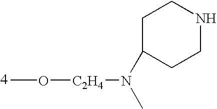 | — |
| 106 | CH₃ | CH₃ | CH₃ | 1 | 3,5 methoxy | 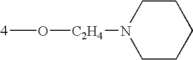 | — |
| 107 | CH₃ | CH₃ | CH₃ | 1 | 3 Cl | 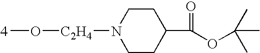 | — |
| 108 | CH₃ | CH₃ | CH₃ | 0 | — | 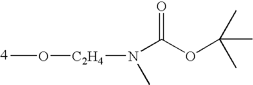 | — |
| 109 | CH₃ | CH₃ | CH₃ | 0 | — | 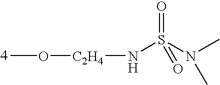 | — |
| 110 | CH₃ | CH₃ | CH₃ | 1 | 3 Cl | 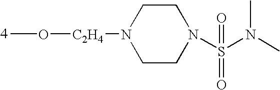 | — |
| 111 | CH₃ | CH₃ | CH₃ | 0 | — | 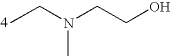 | — |
| 112 | CH₃ | CH₃ | CH₃ | 0 | — | 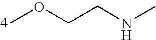 | — |
| 113 | CH₃ | CH₃ | CH₃ | 0 | — | 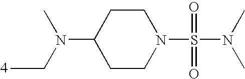 | — |
| 114 | CH₃ | CH₃ | CH₃ | 0 | — | 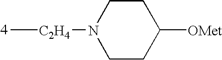 | — |
| 115 | CH₃ | CH₃ | CH₃ | 1 | 3 Cl | 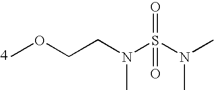 | — |
| 116 | CH₃ | CH₃ | CH₃ | 1 | 3,5 methoxy | 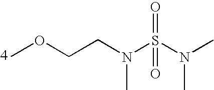 | — |
| 117 | CH₃ | CH₃ | CH₃ | 0 | — | 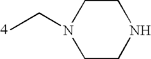 | — |
| 118 | CH₃ | CH₃ | CH₃ | 0 | — | 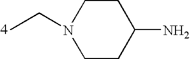 | — |
| 119 | CH₃ | CH₃ | CH₃ | 0 | — | 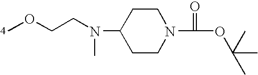 | — |
| 120 | CH₃ | CH₃ | CH₃ | 0 | — | 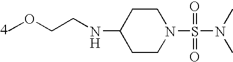 | — |
| 121 | CH₃ | CH₃ | CH₃ | 1 | 3,5 methoxy | 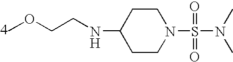 | — |
| 122 | CH₃ | CH₃ | CH₃ | 0 | — | 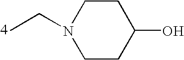 | — |
| 123 | CH₃ | CH₃ | CH₃ | 0 | — | 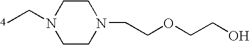 | — |
| 124 | CH₃ | CH₃ | CH₃ | 0 | — | 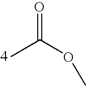 | — |
| 125 | CH₃ | CH₃ | CH₃ | 0 | — | 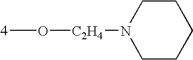 | mp 225° C. |

TABLE 2

| | |
|---|---|
| | (I) |
| 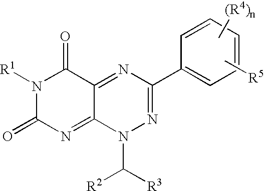 | |

| Co. No. | R¹ | 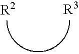 | n | R⁴ | R⁵ | Physical data |
|---|---|---|---|---|---|---|
| 126 | CH₃ | (CH₂)₄ | 0 | — | — | — |
| 127 | CH₃ | (CH₂)₄ | 0 | — | — | mp 240.7- |
| | | | | | | 248.4° C. |
| 128 | CH₃ | (CH₂)₅ | 0 | — | — | mp > 250° C. |
| 129 | CH₃ | (CH₂)₆ | 0 | — | — | mp > 250° C. |
| 130 | CH₃ | (CH₂)₇ | 0 | — | — | mp 227° C. |
| 131 | CH₃ | 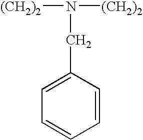 | 0 | — | — | mp 239° C. |
| 132 | CH₃ | 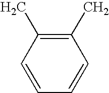 | 0 | — | — | mp > 250° C. |
| 133 | CH₃ | (CH₂)₄ | 0 | — | 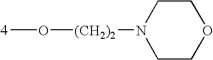 | mp 250° C. |
| 23 | CH₃ | 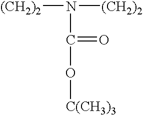 | 0 | — | — | mp 250° C. |
| 24 | CH₃ | 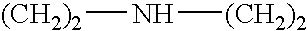 | 0 | — | — | mp > 250° C. |
| 134 | CH₃ | (CH₂)₄ | 0 | — | 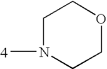 | mp > 250° C. |
| 135 | CH₃ | (CH₂)₄ | 0 | — | 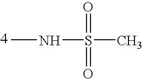 | mp > 250° C. |
| 136 | CH₃ | (CH₂)₄ | 0 | — | 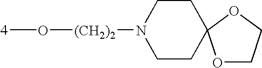 | mp 259° C. |
| 137 | CH₃ | (CH₂)₄ | 0 | — | 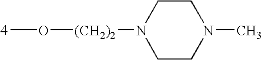 | — |
| 138 | CH₃ | (CH₂)₄ | 0 | — | 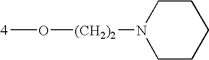 | mp 184.3-223.7° C. |
| 139 | CH₃ | 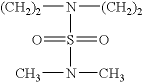 | 0 | — | — | mp 275.9° C. |
| 2 | CH₃ | (CH₂)₄ | 0 | — | 4-CH₂—OH | — |
| 3 | CH₃ | (CH₂)₄ | 0 | — | 4-CH₂—Cl | — |
| 140 | CH₃ | (CH₂)₄ | 0 | — | 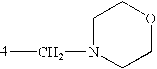 | mp 242.2-245.4° C. |
| 141 | CH₃ | (CH₂)₄ | 0 | — | 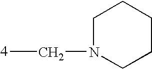 | mp 226.3-297.8° C. |
| 142 | CH₃ | (CH₂)₄ | 0 | — | 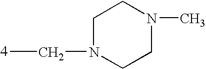 | mp >206.3° C. |
| 143 | CH₃ | (CH₂)₄ | 0 | — | 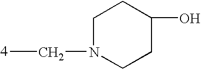 | — |
| 4 | CH₃ | (CH₂)₄ | 2 | 3,5 | 4-OH | — |
| | | | | methoxy | | |
| 25 | CH₃ | (CH₂)₄ | 2 | 3,5 methoxy | 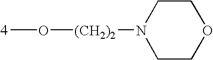 | mp 187.5-209.0° C. |
| 144 | CH₃ | (CH₂)₄ | 1 | 3 methoxy | 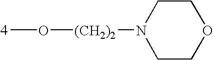 | — |
| 1 | CH₃ | (CH₂)₄ | 1 | 3 | 4-OH | — |
| | | | | methoxy | | |
| 145 | CH₃ | (CH₂)₄ | 0 | — | 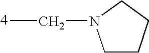 | mp >216.2° C. |
| 146 | CH₃ | (CH₂)₄ | 0 | — | 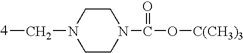 | mp 242.4-260.8° C. |
| 147 | CH₃ | (CH₂)₄ | 0 | — | 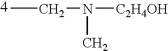 | mp 182.2-213.6° C. |
| 148 | CH₃ | 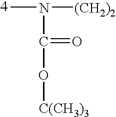 | 0 | — | 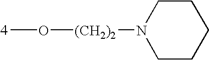 | mp 161.7-210.5° C. |
| 149 | CH₃ | 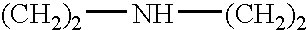 | 0 | 3 methoxy | 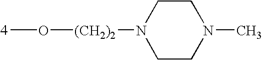 | — |
| 150 | CH₃ | 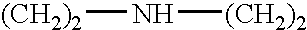 | 0 | — | 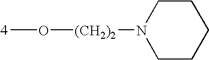 | mp >250° C. |
| 151 | CH₃ | 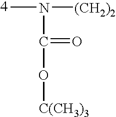 | 1 | 3 methoxy | 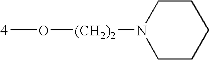 | — |
| 152 | CH₃ | 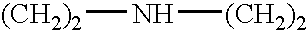 | 1 | 3 methoxy | 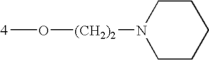 | — |
| 153 | CH₃ | (CH₂)₄ | 0 | — | 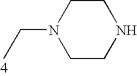 | — |
| 154 | CH₃ | 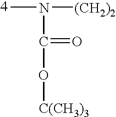 | 0 | — | 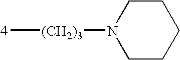 | mp 250° C. |
| 155 | CH₃ | 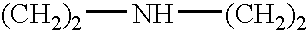 | 0 | — | 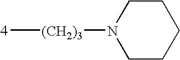 | — |

C. Pharmacological Examples

### EXAMPLE C.1

### In Vitro Inhibition of cdk4 Using a Scintillant Proximity Assay

The scintillant proximity assay (SPA) is in general described in U.S. Pat. No. 4,568,649 (Amersham Pharmacia Biotech). In the present cdk4 SPA kinase reaction assay, a kinase substrate consisting of a fragment of the restinoblastoma protein (pRb) tagged with glutathione-5-transferase (GST), is incubated with the aforementioned protein in the presence of (³³P) radiolabeled ATP. (³³P) phosporylation of the substrate is subsequently measured as light energy emitted using glutathione-coated SPA beads (Amersham Pharmacia Biotech) by trapping and quantifying the binding of the GST tagged and radiolabeled restinoblastoma protein.

### DETAILED DESCRIPTION

The CDK4 SPA kinase reaction is performed at room temperature for 30 minutes in a 96-well microtiter plate. For each of the tested compounds a full dose response −10⁻⁵M to 3.10⁻⁹M—has been performed. Flavopiridol was used as reference compound. The 100 μl reaction volume contains 50 mM Hepes, 10 mM NaF, 10 mM MgCl₂, 1 mM Na₃VO₄ pH 7.5, 1.5 μg CDK4-cell lysate/well, 0.2 μM unlabeled ATP, 1.7 μg/well GST-pRb, 1.7 nM AT³³P and 1 μl of a DMSO solution. The reaction is stopped by diluting the reaction mixture 1/2 with 0.1 mM Na₂EDTA, 0.1 mM non-labeled ATP, 0.05% Triton-X-100 and 10 mg/ml glutathion coated beads in PBS. The microtiterplates are centrifuges at 900 rpm for 10 minutes and the amount of phosphorylated (³³P) pRb is determined by counting (1 min/well) in a microtiterplate scintillation counter.

### EXAMPLE C.2

### In Vitro Inhibition of AKT3 Using a Scintillant Proximity Assay

The scintillant proximity assay (SPA) is in general described in U.S. Pat. No. 4,568,649 (Amersham Pharmacia Biotech). In the present AKT3 SPA kinase reaction assay, a kinase substrate consisting of a fragment of histone H2B tagged with biotine, is incubated with the aforementioned protein in the presence of (³³P) radiolabeled ATP. (³³P) phosporylation of the substrate is subsequently measured as light energy emitted using streptavidine coated SPA beads (Amersham Pharmacia Biotech) by trapping and quantifying the binding of the biotine tagged and radiolabeled histone H2B fragment.

### DETAILED DESCRIPTION

The AKT3 SPA kinase reaction is performed at 25° C. for 3 hrs in a 96-well microtiter plate. For each of the tested compounds a full dose response −10⁻⁵M to 3.10⁻⁹M—has been performed. Staurosporine was used as reference compound [10⁻⁷M to 10⁻⁹M]. The assays were performed in the presence of 25 mM Hepes, pH 7.0, containing 15 mM MgCl₂, 1 mM DTT Each assay was performed in a 100 μl reaction volume containing 111 nM AKT3 (diluted in 25 mM Hepes, pH 7.0, containing 15 mM MgCl₂, 1 mM DTT) and the 0.75 μM Biotinylated Histone H2B and 2 nM ATP-P³³. The reaction was terminated by addition of 100 μl Stop mix (50 μM ATP, 5 mM EDTA, 0.1% BSA, 0.1% Triton X-100 and 7.5 mg/ml Streptavidin coated PVT SPA beads. After allowing the beads to settle for 30 min, the assay mixture was counted in a microtiterplate scintillation counter.

### EXAMPLE C.3

### In Vitro Inhibition of AKT3 Using a Filter Assay

In the present AKT3 filter assay, a kinase substrate consisting of a fragment of histone H2B, is incubated with the aforementioned protein in the presence of (³³P) radiolabeled ATP. The (³³P)phosporylated substrate binds to a phosphocellulose cation exchange filter, that can easily be removed from the incubation mixture and counted using a microplate scintillation counter.

### DETAILED DESCRIPTION

AKT3 filter assays were performed at 25° C. for 3 hrs in the presence of 25 mM Hepes, pH 7.0, containing 15 mM MgCl₂. 1 mM DTT Each assay was performed in a 100 μl reaction volume containing 111 nM AKT3 (diluted in 25 mM Hepes, pH 7.0, containing 15 mM MgCl₂, mM DTT) and the 2.5 μM Histone H2B and 2 nM ATP-P³². The reaction was terminated by addition of 100 μl 75 mM H₃PO₄. 90 μl of the assay mixture was filtered through Phosphocellulose cation exchange paper. After five times washing with 75 μM H₃PO₄, the filterpaper was counting in a microtiterplate scintillation counter.

### EXAMPLE C.4

### Cellular Inhibition of AKT3 Using an ELISA

The human breast adenocarcinoma cell line (MDA-MB 231) was used in an phosphospecific antibody cell ELISA (PACE) to assess the inhibitory effect of the compounds on AKT3 mediated phosphorylation of mitogen-activated protein kinase (MAPK). In the experiments the MDA-MB 231 cells were serum starved for 24 hours (5% CO₂; 37° C.). Subsequently, the cells are incubated at room temperature for 2 hours with 20 μM (in serum free medium) of the phosphatidylinositol 3-kinase inhibitor Ly294002 (Alexis, San Diego, Calif.) prior to the incubation for 30 minutes with the compounds at a final concentration ranging from 1 nM to 3 μM. After fixation (with 4.5% formaldehyde) for 20 minutes and washing with PBS (0.1M) the cells were successively incubated with for 5 minutes with 0.1% Triton X-100 in PBS, for 20 minutes with 0.6% H₂O₂ and 1 hour with a 2% BSA solution as blocking buffer. After overnight incubation with 0.4 fig mouse anti-phospho-MAPK E10 (NEB, #9106) at 4° C., the phosphorylated MAPK was revealed using 0.5 μg anti mouse IgG HRP (Promega, #W402B) as secondary antibody followed by a 15 minutes incubation using OPD (Sigma, #8287) as a detection buffer. The OD (490-655 nm) reflected the amount of phosphorylated MAPK and the pIC₅₀ of the compounds was based on their effect with respect to blanco (0.1% DMSO) or an internal reference compound treatment.

### EXAMPLE C.5

### In Vitro Inhibition of CDC25B Using the Fluorogenic Substrate 3-OMFP

CDC25B phosphatase activity is assessed using the fluorogenic substrate 3-O-methyl-flurorescein-phosphate (3-OMFP). The phosphatase-reaction is performed for 1 hour at room temperature in a black microtiter plate in a volume of 50 μl. The reaction mixture contains 4 μg/mlCDC25B, 15 μM (3-OMFP), 15 mM Tris, 50 mM NaCl, 1 mM DTT, 1 mM Na₂EDTA at pH 8.0 and 0.1% DMSO solution at 10⁻⁵ M and the hits are tested in the same conditions in a full dose/response from 10⁻⁵, 3.10⁻⁶, 10⁻⁶ and 3.10⁻⁷ M. The enzymatic activity is determined by measuring the fluorescent signal at 485 nm (ex.) and 538 (em.).

### EXAMPLE C.6

### Cellular Inhibition of AKT3 Using an ELISA

The human breast adenocarcinoma cell line (MDA-MB 231) was used in an phosphospecific antibody cell ELISA (PACE) to assess the inhibitory effect of the compounds on AKT3 mediated phosphorylation of mitogen-activated protein kinase (MAPK). In the experiments the MDA-MB 231 cells were serum starved for 24 hours (5% CO₂; 37° C.). Subsequently, the cells are incubated at room temperature for 2 hours with 20 μM (in serum free medium) of the phosphatidylinositol 3-kinase inhibitor Ly294002 (Alexis, San Diego, Calif.) prior to the incubation for 30 minutes with the compounds at a final concentration ranging from 1 nM to 3 μM. After fixation (with 4.5% formaldehyde) for 20 minutes and washing with PBS (0.1M) the cells were successively incubated with for 5 minutes with 0.1% Triton X-100 in PBS, for 20 minutes with 0.6% H₂O and 1 hour with a 2% BSA solution as blocking buffer. After overnight incubation with 0.4 μg mouse anti-phospho-MAPK E10 (NEB, #9106) at 4° C., the phosphorylated MAPK was revealed using 0.5 μg anti mouse IgG HRP (Promega, #W402B) as secondary antibody followed by a 15 minutes incubation using OPD (Sigma, #8287) as a detection buffer. The OD (490-655 nm) reflected the amount of phosphorylated MAPK and the pIC₅₀ of the compounds was based on their effect with respect to blanco (0.1% DMSO) or an internal reference compound treatment.

In the following table, cross kinase activity with improved solubility is demonstrated for the compounds according to the invention.

| | Solubility pH 7.4: | | | | | |
|---|---|---|---|---|---|---|
| | Class 2: (1.8 × | CDK4 SPA | AKT3 pep. | AKT cel | Cytotox survival of A2780 | CDC25B |
| Compound | 10−3M-1.6 × 10−4M) | (Ex. C.1): pIC50 | (Ex. C.3): pIC50 | (Ex. C.6): pIC50 | cells after 3 days - pIC50 | WT (Ex. C.5): pIC50 |
| number | Class 3: (>1.8 × 10−3M) | values | values | values | values | values |
| 74 | 2 (stock 5 mM) | 6.793 | 6.956 | NT | 6.721 | NT |
| 148 | 2 (stock 5 mM) | 6.877 | 7.062 | NT | 6.873 | 6.928 |
| 151 | 2 (stock 5 mM) | 6.75 | 6.843 | <6.523 | 6.719 | 7.661 |
| 77 | 3 (stock 5 mM) | 6.533 | 6.619 | <6 | 6.785 | 7.364 |
| 78 | 3 (stock 5 mM) | 6.29 | 6.581 | 6.521 | 6.570 | 7.357 |
| 79 | 2 (stock 5 mM) | 6.394 | 6.549 | 5.523 | 6.290 | 7.582 |
| 80 | 2 (stock 5 mM) | 6.43 | 6.403 | <6.523 | 6.409 | 7.489 |
| 81 | 3 (stock 5 mM) | 6.713 | 6.599 | <6 | 6.879 | 7.41 |
| 152 | 3 (stock 5 mM) | 6.728 | 6.56 | 5.523 | 6.201 | 7.595 |
| 82 | 3 (stock 5 mM) | 6.523 | 6.47 | 6.666 | 6.426 | 7.299 |
| 153 | 3 (stock 5 mM) | 6.433 | 6.475 | 6.305 | 6.742 | 7.337 |
| 84 | 3 (stock 5 mM) | 6.232 | 6.553 | <6 | 6.666 | 7.417 |
| 85 | 3 (stock 5 mM) | 6.492 | 6.462 | <6 | 6.642 | 7.315 |
| 86 | 2 (stock 5 mM) | 6.566 | 6.564 | <6 | 6.578 | 7.413 |
| 87 | 2 (stock 5 mM) | 6.562 | 6.387 | <6 | 6.703 | 7.316 |
| 88 | 2 (stock 5 mM) | 6.573 | 6.622 | <6 | 6.775 | 7.589 |
| 89 | 3 (stock 5 mM) | 6.296 | 6.454 | <6 | 6.278 | 7.45 |
| 90 | 3 (stock 5 mM) | 6.691 | 6.743 | <6 | 6.842 | 7.555 |
| 93 | 3 (stock 5 mM) | 6.714 | 6.554 | <6 | 6.565 | 7.309 |
| 94 | 3 (stock 5 mM) | 6.762 | 6.782 | NT | 6.788 | 7.439 |
| 95 | 2 (stock 5 mM) | 6.493 | 6.721 | <5.523 | 6.318 | 7.383 |
| 96 | 3 (stock 5 mM) | 6.689 | 6.757 | <5.523 | 6.308 | 7.347 |
| 97 | 3 (stock 5 mM) | 6.786 | 6.704 | <5.523 | 6.535 | 7.37 |
| 98 | 3 (stock 5 mM) | 6.7 | 6.713 | <5.523 | 6.312 | 7.233 |
| 99 | 3 (stock 5 mM) | 6.85 | 6.769 | 6.365 | 6.921 | 7.519 |
| 100 | 3 (stock 5 mM) | 6.803 | 6.75 | 6.39 | 6.609 | 7.302 |
| 155 | 3 (stock 5 mM) | 7.139 | 6.634 | 6.329 | 6.752 | 7.433 |
| 101 | 3 (stock 5 mM) | 6.638 | 6.635 | 6.29 | 6.237 | 7.272 |
| 102 | 3 (stock 5 mM) | 7.098 | 6.764 | <5.52 | 6.040 | 7.438 |
| 103 | 2 (stock 5 mM) | 6.447 | 6.888 | <5.52 | 6.372 | 7.646 |
| 104 | 3 (stock 5 mM) | 6.894 | 6.919 | 6.139 | 6.670 | 7.52 |
| 105 | 3 (stock 5 mM) | 6.815 | 6.86 | <5.52 | >5.522 | 7.449 |
| 106 | 3 (stock 5 mM) | 6.849 | 6.932 | <5.52 | 6.074 | 7.478 |
| 109 | 2 (stock 5 mM) | 6.779 | 6.81 | <5.52 | 6.226 | NT |
| 111 | 3 (stock 5 mM) | 6.9 | 6.792 | 6.102 | 6.804 | 7.509 |
| 113 | 3 (stock 5 mM) | 6.821 | 6.735 | <5.52 | 6.658 | 6.924 |

D. Composition Examples

The following formulations exemplify typical pharmaceutical compositions suitable for systemic administration to animal and human subjects in accordance with the present invention.

“Active ingredient” (A.I.) as used throughout these examples relates to a compound of formula (I) or a pharmaceutically acceptable addition salt thereof.

### EXAMPLE D.1

### Film-Coated Tablets

Preparation of Tablet Core

A mixture of A.I. (100 g), lactose (570 g) and starch (200 g) was mixed well and thereafter humidified with a solution of sodium dodecyl sulfate (5 g) and polyvinyl-pyrrolidone (10 g) in about 200 ml of water. The wet powder mixture was sieved, dried and sieved again. Then there was added microcrystalline cellulose (100 g) and hydrogenated vegetable oil (15 g). The whole was mixed well and compressed into tablets, giving 10.000 tablets, each comprising 10 mg of the active ingredient.

Coating

To a solution of methyl cellulose (10 g) in denaturated ethanol (75 ml) there was added a solution of ethyl cellulose (5 g) in CH₂Cl₂ (150 ml). Then there were added CH₂Cl₂ (75 ml) and 1,2,3-propanetriol (2.5 ml). Polyethylene glycol (10 g) was molten and dissolved in dichloromethane (75 ml). The latter solution was added to the former and then there were added magnesium octadecanoate (2.5 g), polyvinyl-pyrrolidone (5 g) and concentrated color suspension (30 ml) and the whole was homogenated. The tablet cores were coated with the thus obtained mixture in a coating apparatus.

1. A compound having the formula 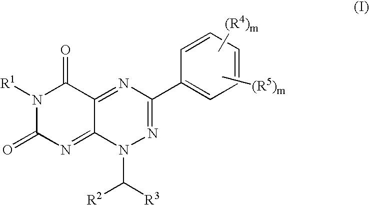
the N-oxide forms, the pharmaceutically acceptable addition salts and the stereo-chemically isomeric forms thereof, wherein
n represents an integer being 0, 1 or 2;
m represents an integer being 0 or 1;
R¹ represents hydrogen, Ar¹, C₁₋₄alkyl or C₁₋₄alkyl substituted with morpholinyl or pyridinyl;
R² represents hydrogen, phenyl, C₁₋₄alkyl, C₁₋₄alkyloxycarbonyl or C₁₋₄alkyl substituted with hydroxy, phenyl or -oxy-halophenyl;
R³ represents hydrogen, phenyl, C₁₋₄alkyl, C₁₋₄alkyloxycarbonyl or C₁₋₄alkyl substituted with hydroxy, phenyl or -oxy-halophenyl; or
R² and R³ taken together with the carbon atom to which they are attached form a C₃₋₈cycloalkyl or Het¹ wherein said C₃₋₈cycloalkyl or Het¹ each independently may optionally be substituted with one, or where possible, two or three substituents each independently selected from C₁₋₄alkyloxycarbonyl, —C₁₋₄alkyl-Ar³ C₁₋₄alkylsulfonyl, aminosulfonyl, mono- or di(C₁₋₄alkyl)aminosulfonyl or —C(═NH)—NH₂;
R⁴ represents halo, nitro, hydroxy or C₁₋₄alkyloxy;
R⁵ represents formyl, hydroxy, cyano, phenyl, —O—Ar², NR⁶R⁷, C₁₋₄alkyl, C₁₋₄alkyloxy, C₁₋₄alkylsulfonyl, C₁₋₄alkylcarbonyl, C₁₋₄alkyloxycarbonyl, —O-(mono- or di(C₁₋₄alkyl)aminosulfonyl), Het², —SO₂-Het⁶, C₂₋₆alkenyl optionally substituted with phenyl,
C₁₋₄alkyl substituted with one or where possible more substituent being selected from hydroxy, halo, Het³, NR⁶R⁷ or formyl,
C₁₋₄alkyloxy substituted with one or where possible more substituents being selected from halo, amino, mono- or di(C₁₋₄alkyl)aminosulfonyl, aminosulfonyl, Het⁴, NR⁸R⁹ or —C(═O)-Het⁴;
R⁶ and R⁷ are each independently selected from hydrogen, C₁₋₄alkyl,
C₁₋₄alkyloxyC₁₋₄alkyl, Het⁵ or C₁₋₄alkyl substituted with one or where possible more substituents being selected from hydroxy, Het⁵, C₁₋₄alkyloxycarbonyl, or
C₁₋₄alkylsulfonyl;
R⁸ and R⁹ are each independently selected from hydrogen, C₁₋₄alkyl, C₁₋₄alkyloxycarbonyl, Het⁷, mono- or di(C₁₋₄alkyl)aminosulphonyl or aminosulphonyl;
Het¹ represents piperidinyl or dihydroindenyl;
Het² represents a heterocycle selected from piperidinyl, morpholinyl, or piperazinyl wherein said monocyclic heterocycles each independently may optionally be substituted with one, or where possible two or three substituents each independently selected from C₁₋₄alkyloxycarbonyl;
Het³ represents a heterocycle selected from morpholinyl, pyrrolidinyl, pyrrolyl, piperidinyl, or piperazinyl wherein said monocyclic heterocycles each independently may optionally be substituted with one, or where possible two or three substituents each independently selected from hydroxy, C₁₋₄alkyl, C₁₋₄alkyloxycarbonyl, hydroxyC₁₋₄alkyl, aminosulfonyl, NR¹⁰R¹¹, imidazolyl, tetrahydropyrimidinyl, amino, mono- or di(C₁₋₄alkyl)aminosulfonyl, hydroxyC₁₋₄alkyloxyC₁₋₄alkyl, C₁₋₄alkyloxyC₄alkyl or C₁₋₄alkyloxy;
R¹⁰ and R¹¹ are each independently selected from hydrogen, C₁₋₄alkyl,
C₁₋₄alkyloxycarbonyl, aminosulfonyl, or mono- or di(C₁₋₄alkyl)aminosulfonyl;
Het⁴ represents a heterocycle selected from morpholinyl, piperidinyl, imidazolyl or piperazinyl wherein said monocyclic heterocycles each independently may optionally be substituted with one, or where possible two or three substituents each independently selected from hydroxy, C₁₋₄alkyl, C₁₋₄alkyloxycarbonyl, aminosulfonyl or mono- or di(C₁₋₄alkyl)aminosulfonyl or Het⁴ represents a monovalent radical represented by formula (i); 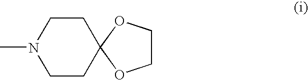
Het⁵ represents a heterocycle selected from pyridinyl, pyrimidinyl, pyrrolidinyl, or piperidinyl wherein said monocyclic heterocycles each independently may optionally be substituted with one, or where possible two or three substituents each independently selected from C₁₋₄alkyl, C₁₋₄alkyloxycarbonyl, aminosulfonyl, C₁₋₄alkylaminosulfonyl or mono- or di(C₁₋₄alkyl)aminosulfonyl;
Het⁶ represents morpholinyl;
Het⁷ represents pyridinyl, piperidinyl, piperazinyl or pyrimidinyl optionally substituted with C₁₋₄alkylphenyl, C₁₋₄alkyloxycarbonyl aminosulfonyl, or mono- or di(C₁₋₄alkyl)aminosulfonyl;
Ar¹ represents an aryl substituent selected from phenyl or naphthalenyl wherein said aryl substituents each independently may optionally be substituted with one, or where possibly two or three substituents each independently selected from nitro or C₁₋₄alkyloxycarbonyl;
Ar² represents phenyl optionally substituted with one or where possible two or three substituents each independently selected from the group consisting of halo and nitro;
Ar³ represents an aryl substituent selected from the group consisting of phenyl,

2. A compound according to claim 1 wherein;
R¹ represents Ar¹, C₁₋₄alkyl preferably methyl, or C₁₋₄alkyl substituted with morpholinyl;
R² represents hydrogen or C₁₋₄alkyl;
R³ represents hydrogen or C₁₋₄alkyl; or
R² and R³ taken together with the carbon atom to which they are attached form a C₃₋₈cycloalkyl or Het¹ wherein said C₃₋₈cycloalkyl or Het¹ each independently may optionally be substituted with C₁₋₄alkyloxycarbonyl;
R⁴ represents halo preferably chloro or R⁴ represents C₁₋₄alkyloxy preferably methoxy;
R⁵ represents C₁₋₄alkyloxycarbonyl, —O-(mono- or di(C₁₋₄alkyl)aminosulfonyl),
C₁₋₄alkyl substituted with one or where possible more substituent being selected from Het³ or NR⁶R⁷,
C₁₋₄alkyloxy substituted with one or where possible more substituents being selected from amino, Het⁴ or NR⁸R⁹;
R⁶ and R⁷ are each independently selected from hydrogen, C₁₋₄alkyl,
C₁₋₄alkyloxyC₁₋₄alkyl, Het⁵ or C₁₋₄alkyl substituted with one or where possible more substituents being selected from hydroxy or Het⁵;
R⁸ and R⁹ are each independently selected from hydrogen, C₁₋₄alkyl, C₁₋₄alkyloxycarbonyl, Het⁷ or mono- or di(C₁₋₄alkyl)aminosulphonyl;
Het¹ represents piperidinyl;
Het³ represents a heterocycle selected from morpholinyl, pyrrolidinyl, piperidinyl, or piperazinyl wherein said monocyclic heterocycles each independently may optionally be substituted with one, or where possible two or three substituents each independently selected from hydroxy, C₁₋₄alkyl, aminosulfonyl, amino, mono- or di(C₁₋₄alkyl)aminosulfonyl, hydroxyC₁₋₄alkyloxyC₁₋₄alkyl or C₁₋₄alkyloxy;
Het⁵ represents pyridinyl optionally substituted with mono- or di(C₁₋₄alkyl)aminosulfonyl;
Het⁷ represents piperidinyl optionally substituted with C₁₋₄alkylphenyl, C₁₋₄alkyloxycarbonyl, or mono- or di(C₁₋₄alkyl)aminosulfonyl;
Ar¹ represents an aryl substituent selected from phenyl or naphthalenyl;

3. A compound according to claim 1 wherein;
R¹ represents C₁₋₄alkyl preferably methyl;
R² represents C₁₋₄alkyl preferably methyl;
R³ represents C₁₋₄alkyl preferably methyl; or
R² and R³ taken together with the carbon atom to which they are attached form a C₃₋₈cycloalkyl preferably cyclopentyl or Het¹ preferably piperidinyl wherein said C₃₋₈cycloalkyl or Het¹ each independently may optionally be substituted with C₁₋₄alkyloxycarbonyl preferably t-butoxycarbonyl;
R⁴ represents halo or C₁₋₄alkyloxy;
R⁵ represents C₁₋₄alkyloxycarbonyl, —O-(mono- or di(C₁₋₄alkyl)aminosulfonyl),
C₁₋₄alkyl substituted with one or where possible more substituent being selected from Het³ or NR⁶R⁷,
C₁₋₄alkyloxy substituted with one or where possible more substituents being selected from amino, Het⁴ or NR⁸R⁹;
R⁶ and R⁷ are each independently selected from hydrogen, C₁₋₄alkyl, C₁₋₄alkyloxyC₁₋₄alkyl, -Het⁵ or C₁₋₄alkyl substituted with one or where possible more substituents being selected from hydroxy, or Het⁵;
R⁸ and R⁹ are each independently selected from hydrogen, C₁₋₄alkyl, -Het⁷ or mono- or di(C₁₋₄alkyl)aminosulphonyl;
Het³ represents a heterocycle selected from piperidinyl, or piperazinyl wherein said monocyclic heterocycles each independently may optionally be substituted with one, or where possible two or three substituents each independently selected from hydroxy, aminosulfonyl, amino, mono- or di(C₁₋₄alkyl)aminosulfonyl, hydroxyC₁₋₄alkyloxyC₁₋₄alkyl or C₁₋₄alkyloxy;
Het⁴ represents a heterocycle selected from morpholinyl, piperidinyl or piperazinyl wherein said monocyclic heterocycles each independently may optionally be substituted with one, or where possible two or three substituents each independently selected from C₁₋₄alkyl, C₁₋₄alkyloxycarbonyl or mono- or di(C₁₋₄alkyl)aminosulfonyl;
Het⁵ represents a heterocycle selected from pyridinyl or piperidinyl wherein said monocyclic heterocycles each independently may optionally be substituted with one, or where possible two or three substituents each independently selected from aminosulfonyl, or mono- or di(C₁₋₄alkyl)aminosulfonyl;
Het⁷ represents piperidinyl.

4. A compound as claimed claim 1 in any one of claims 1 to 3 wherein R² and R³ taken together with the carbon atom to which they are attached form a C₃₋₈cycloalkyl, preferably cyclopentyl.

5. A compound according to claim 1 wherein R⁵ represents formyl, hydroxy, cyano, phenyl, —O—Ar², NR⁶R⁷, C₁₋₄alkylsulfonyl, C₁₋₄alkylcarbonyl, C₁₋₄alkyloxycarbonyl, —O-(mono- or di(C₁₋₄alkyl)aminosulfonyl), Het², —SO₂-Het⁶, C₂₋₆alkenyl optionally substituted with phenyl,
C₁₋₄alkyl substituted with one or where possible more substituent being selected from hydroxy, halo, Het³, NR⁶R⁷ or formyl, or
C₁₋₄alkyloxy substituted with one or where possible more substituents being selected from halo, amino, mono- or di(C₁₋₄alkyl)aminosulfonyl, aminosulfonyl, Het⁴, NR⁸R⁹ or —C(═O)-Het⁴;

6. A compound according to claim 11 provided that when R⁵ represents NR⁶R⁷, either R⁶ or R⁷ represents C₁₋₄alkylsulfonyl or C₁₋₄alkylcarbonyl, preferably methylsulfonyl or methylcarbonyl.

7. A compound as claimed in claim 1, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.

8. A pharmaceutical composition comprising a pharmaceutically acceptable carrier and, as active ingredient, an effective kinase inhibitory amount of a compound as described in claim 1.

9. A process of preparing a pharmaceutical composition as defined in claim 8, comprising a pharmaceutically acceptable carrier is intimately mixed with an effective kinase inhibitory amount of a compound as described in claim 1.

10. (canceled)

11. (canceled)

12. A process of preparing a compound as described in claim 1, comprising
i) reacting a primary amine of formula (V) with an aldehyde of formula (VI) in a condensation reaction using ethanol as a suitable solvent; 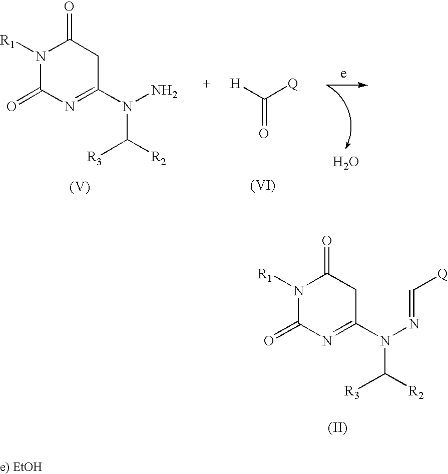
ii) followed by a nitrosative cyclisation of the thus obtained Schiffs bases of formula (II) with NaNO₂ in acetic acid, and refluxing the nitroso intermediates of formula (III) in a suitable solvent such as acetic anhydride or ethanol further comprising dithiothreitol (DTT);

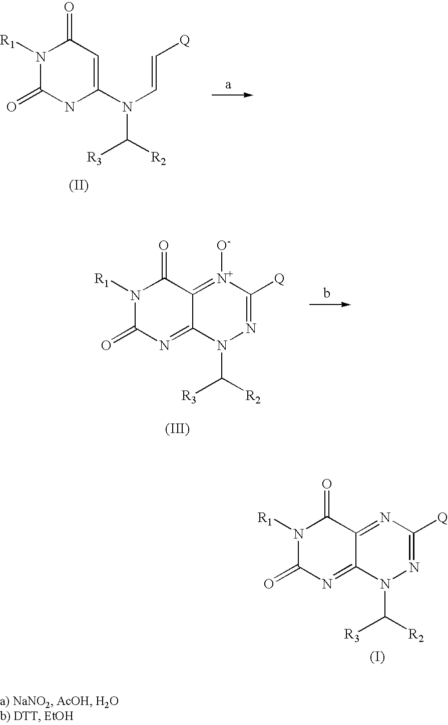

13. A compound as claimed claim 2, wherein R² and R³ taken together with the carbon atom to which they are attached form a C₃₋₈cycloalkyl, preferably cyclopentyl.

14. A compound as claimed claim 3, wherein R² and R³ taken together with the carbon atom to which they are attached form a C₃₋₈cycloalkyl, preferably cyclopentyl.

15. A compound according to claim 2, provided that when R⁵ represents NR⁶R⁷, either R⁶ or R⁷ represents C₁₋₄alkylsulfonyl or C₄alkylcarbonyl, preferably methylsulfonyl or methylcarbonyl.

16. A compound according to claim 3, provided that when R⁵ represents NR⁶R⁷, either R⁶ or R⁷ represents C₁₋₄alkylsulfonyl or C₁₋₄alkylcarbonyl, preferably methylsulfonyl or methylcarbonyl.

17. A compound according to claim 4, provided that when R⁵ represents NR⁶R⁷, either R⁶ or RP represents C₁₋₄alkylsulfonyl or C₁₋₄alkylcarbonyl, preferably methylsulfonyl or methylcarbonyl.

18. A compound according to claim 5, provided that when R⁵ represents NR⁶R⁷, either R⁶ or R⁷ represents C₁₋₄alkylsulfonyl or C₁₋₄alkylcarbonyl, preferably methylsulfonyl or methylcarbonyl.

19. A compound as claimed in claim 2, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.

20. A compound as claimed in claim 2, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.

21. A compound as claimed in claim 3, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.

22. A compound as claimed in claim 4, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.

23. A compound as claimed in claim 5, provided that when R⁵ represents a C₁₋₄alkyloxy substituted Het⁴, said Het⁴ being selected from the group consisting of morpholinyl, piperidinyl, piperazinyl and piperazinyl substituted with one C₁₋₄alkyl substituent, preferably methyl, more preferably with the methyl in the para position relative to the carbon atom bearing the R⁵ substituent, or Het⁴ consists of piperazinyl substituted with one mono- or di(C₁₋₄alkyl)aminosulfonyl substituent, preferably dimethylaminosulfonyl, more preferably with the dimethylaminosulfonyl in the para position relative to the carbon atom bearing the R⁵ substituent.
